(19) United States
(12) Patent Application Publication
(10) Pub. No.: US 2020/0352917 A1
(43) Pub. Date: Nov. 12, 2020

(54) MEDICATED SPRAY FOR TREATMENT OF SUBSTANCE ABUSE, OVERDOSE, ADDICTION AND IMPULSE CONTROL DISORDERS

(71) Applicant: Lance L. GOOBERMAN, Merchantville, NJ (US)

(72) Inventor: Lance L. GOOBERMAN, Merchantville, NJ (US)

(21) Appl. No.: 16/404,462

(22) Filed: May 6, 2019

(51) Int. Cl. A61K 31/439; A61K 9/00

(52) U.S. Cl. CPC A61K 31/439; A61K 9/0078

(57) ABSTRACT

A method to treat carfentanyl overdose comprising administrating a pharmaceutical formulation in the form of liquid solution for spray administration by the nasal and/or buccal route containing naltrexone as active ingredient in amounts greater than 1%.

### FIELD OF THE INVENTION

The present invention is directed to portable application devices and compositions for nasal and oral delivery of substances and/or compositions to treat substance overdose, addiction and/or behavioral disordered persons, and particularly as an effective on demand apparatus, composition and method to treat possible substance abuse overdoses and/or to provide a readily available antidote to various substances which are or may oftentimes prove dangerous, and potentially fatal.

### BACKGROUND OF THE INVENTION

Many substance and/or behavioral addicted persons have experienced much difficulty with impulse control and oftentimes engage in unhealthy or unproductive self medication, sonic of which may be prescribed but yet ineffective or impractical. For example, there are both nicotine substitutes/replacements available without a prescription and replacements which do require a prescription. These include lozenges, patches, gum, sprays and electronic cigarettes or other simulated smoking devices.

Heroin use and addiction has become a major problem approaching epidemic proportions in recent times, compounded by the availability of synthetic opiates many times more potent than heroin leading to an unprecedented number of overdoses and deaths.

Many heroin treatment regimens are also problematic including, for example, substitute medications for opiates, such as heroin or Oxycontin®, include Buprenorphine, sold under the brands of Suboxone® and Subutex® and methadone available as Dolophine® Methadose® and Methadone Diskets®. Methadone is a tightly controlled substance available only from licensed methadone clinics, whereas Buprenorphine is available by prescription at many pharmacies. Buprenorphine is a controlled substance and only available through specifically licensed entities.

The goal on many of the substitutes and replacements for the original substance i.s to provide a replacement that is not nearly as enjoyable as the original, but one that prevents highly unpleasant withdrawal symptoms from occurring. For example, Buprenorphine is a partial opiate antagonist, or otherwise possess a drug or medication efficacy which stimulates activity of opiate receptors in the brain, but yet does not produce as strong an effect as a full opiate agonist such as morphine, methadone, oxycodone, hydrocodone, heroin and codeine, like full opioid agonists, dissimulation occurs at receptors which are normally stimulated by naturally occurring opioids called endorphins. Buprenorphine by displacing agonists from receptors and preventing re-attachment and has the effect of reducing withdrawal symptoms and suppressing cravings, yet it does not have the same strong pleasure characteristic of faster acting opiate drugs Buprenorphine also has a longer withdrawal period than other shorter acting opiates. Because Buprenorphine has a very long half life or the half life of how long it takes the body to break down and eliminate the presence of a drug, it remains in the system relatively longer than other agonists and does not need to be taken as frequently which has the benefit of helping to reduce cravings, suppress unpleasant withdrawal symptoms and break the habit of a quicker more repetitive administration of a faster acting opiate drug or the abuse of such. Notwithstanding, buprenorphine itself often becomes an addictive problem.

Methadone, on the other hand, is a full opiate agonist and mimics the pharmacological activity of opiates such as heroin. Methadone differs from heroin in its pharmacokinetics in that it is an oral medication that must pass through the digestive track slowing its excess to the brain relative to heroin but lasting much longer than heroin. In this way methadone is useful for preventing withdrawal symptoms and is thought to allow people to taper their heroin use down gradually or their methadone use down gradually without producing the expected sought after more extreme high associated with a faster acting opiate agonist. However, as with buprenorphine, methadone is also a problem addictive compound due, inter alia, a long half-life.

As to further examples of addiction treatment, nicotine replacement therapy (NRT) comes in several forms. Skin patches (Habitrol® and Nicoderm®), gum (Nicorette®), and lozenges (Commit®) deliver controlled doses of nicotine. These delivery methods are available over the counter (OTC), without prescription. The nasal spray and inhaler are available by prescription (Nicotrol®). These products replace nicotine but eliminate the toxins in tobacco combustion products. Many generic products for NRT are also available.

Another known method is to attempt to restore healthy neurophysiological functioning such so that people do not use addictive substances or activities as a means for self-medication, or to correct neurophysiological damage resulting from chronic drug use, or addictive activities.

Acamprosate (Camprol®) is used to treat alcohol abuse, and is also known to decrease some of the physical and psychological symptoms associated with alcohol withdrawl. Acamprosate is believed to restore the chemical balance in a brain that became unbalanced by chronic alcohol use. While remaining an FDA-approved medication, recent studies now doubt its effectiveness.

Buproprion (Zyban®, Wellbutrin® Voxra®, and Budeprion®) are drugs that are used to target relapse prevention for tobacco withdrawal. Nicotine is the addictive drug in tobacco products like cigarettes, cigars, and chewing tobacco. This medication is in the class of atypical antidepressants. These medications block the reuptake (reabsorption) of the neurotransmitters dopamine and norepinephrine. Reuptake blockers work by allowing neurotransmitters to remain in the synapse for a longer period. This permits them to bind to more receptors. Re-establishing activity in neurotransmitter systems enhances the recovery process. Bupropione, originally used as an antidepressant, has lately been used in a compound called Contrave®, a combination of bupropione and naltrexone, and is used to treat obesity stemming from, inter alia food and/or carbohydrate addiction.

Still other example medications contemplated for use herein are: Exonatide (Byetta® and Bydureon®) an injectable used to treat diabetes, and liraglutide (Victoza® and Saxenda®) also used in diabetes treatment in addition to other dulaglutides such as Trulicity®.

Varenicline (Chantix®) is also used to treat tobacco use disorder or addiction, and is known as a partial agonist of the nicotine receptor. It's unclear exactly how this compound works, but appears to suppress cravings associated with nicotine use by stimulating the brain's reward system.

Benzodiazepines (e.g. Xanax®, Librium®, Ativan®, Klonopin®, Diazepam/Valium®) are often called anti-anxiety drugs or anxiolytics, and are thought by many as the most controversial in addiction treatment as many people become addicted to these drugs as they are frequently abused (see Sedative, Hypnotic, or Anxiolytic Use Disorders). This class of drugs works by activating the GABA receptors in the brain to relieve the anxiety associated with withdrawal. They also can compensate for changes that occur in the GABA system following withdrawal from sedative drugs such as alcohol or opiates. Furthermore, some people may abuse alcohol or opiates because they are attempting to self-medicate a pre-existing anxiety disorder. Anti-anxiety drugs are also useful for treating these underlying disorders. Anxiety is also a symptom of many other types of psychiatric disorders. To some, the cautious and judicious use of benzodiazepines may be helpful, particularly during, the early stages of recovery, but addiction to benzodiazepines, as mentioned, remains a concern.

Conventional treatment also includes the prevention or diminishment of powerful cravings that cause people to resume drug use or an addictive activity, after a period of cessation.

Naltrexone has several recognized uses, and in some applications may be useful for those aiming to moderate alcohol consumption, and/or to treat sex addiction, gambling urges, internet pornography, pedophilia, and any and all other activities that involve the dopaminergic system, or the so-called “reward” brain system. One of naltrexone's known primary functions is to suppress alcohol craving. In other applications, a preferred embodiment herein, naltrexone and other opiate antagonists, such as a naloxone, are useful in controlling opiate use urges, and even more importantly combating potential overdose.

In recent years synthetic opiates (opioids) such as fentanyl and especially the myriad of fentanyl analogs appearing in illicit drug sales and distribution have presented particularly challenging and pressing problems. Fentanyl known for some time is many times stronger, more potent than morphine, and heroin, and many analogs of fentanyl are hundreds, or even thousands of times more potent leading to current epidemic of drug overdose deaths with no known treatment.

Keegan et al. of US2017/0071851 discloses drug products adapted for nasal delivery, comprising a device filled with a pharmaceutical composition, and which may comprise opioid antagonist, specifically naloxone only, for treating fentanyl and fentanyl analog overdose. However, literature reports that naloxone in fact cannot reverse fentanyl and/or fentanyl analog overdose. See Fentanyl Found in Georgia Resists Lprife-Saving Naloxone Antidote—by Jessica Firger on Jun. 28, 2017.

Thus, to this end there is seen a need for the convenient and sometimes spontaneous application by nasal and/or oral delivery of substances and compositions to treat or self treat as needed substance addicted and/or behavioral disordered persons on demand, and possible substance abuse overdoses. There exists an imminent need for fentanyl and fentanyl overdose treatment and reversal, as none prior to this invention exist.

### SUMMARY OF THE INVENTION

In its broadest and most important aspect, the invention provides novel apparatus and compositions in the form of a spray applicator for administering nasally or orally for fentanyl analogues overdose and reversal treatment, for which no known treatment has heretofore existed. There currently exists a panic and epidemic of fentanyl and fentanyl analog overdose deaths for which the invention provides a solution. In other aspects, the invention provides apparatus and compression in the form of spray for an anti-addiction and/or behavioral treatment and/or overdose treatment or prevention substance selected from any of the above described substances in the background of the invention, and/or any other such known (or yet unknown) anti-addiction and/or behavioral treatment and/or overdose treatment or prevention substances, and in preferred embodiments an opiate antagonist selected from naloxone and/or naltrexone. The applicator is capable of delivering metered, calibrated single or multiple doses of the anti-addiction and/or behavioral treatment and/or overdose treatment or prevention substance, such as an opiate antagonist, on demand through a projecting delivery portion which is shaped or dimensioned for introduction into the nose or mouth. A pharmaceutical composition for nasal or oral administration is also disclosed which may comprise, for example, an addiction antagonist and/or behavioral treatment and/or overdose treatment or prevention substance, preferably an opioid antagonist such as naloxone and/or naltrexone, and which may comprise a water-susceptible solid carrier admixed with the opioid antagonist.

### DESCRIPTION OF DRAWINGS

FIG. 1 shows schematic drawings of fentanyl and major fentanyl analogs

FIG. 2a shows schematic drawing of synthesis of fentanyl

FIG. 2b shows schematic drawings of Structures of diampromide (1S) and 2,3-seco-fentanyl (2S)

FIG. 3a shows schematic drawings of modifications of the structure of fentanyl

FIG. 3b shows schematic drawing of structures of conformationally restricted, semirigid analogues of fentanyl

FIG. 4a shows schematic drawing of synthesis of perhydroazepine analogs of fentanyl

FIG. 4b shows schematic drawing of synthesis of 2-methyl-fentanyl

FIG. 5a shows schematic drawings of methyl-substituted fentanyls

FIG. 5b shows schematic drawing of synthesis of 3-methyl-fentanyl

FIG. 6a shows schematic drawing of synthesis of 4-aryl-fentanyl analogs

FIG. 6b shows schematic drawing of structures of α-methylfentanyl and its 3-methyl analogue

FIG. 7a shows schematic drawing of synthesis of carfentanyl, alfentanil sufentanil and their analogs

FIG. 7b shows schematic drawing of synthesis of trans-3-alkylfentanyls

FIG. 8a shows schematic drawing of alternative synthesis of 4-aryl-fentanyls from corresponding acids

FIG. 8b shows schematic drawing of synthesis of 2.5-dimethyl-fentanyl-phenaridine

FIG. 9a shows schematic drawing of synthesis of 4-acloxymethyl-fentanyls

FIG. 9b shows schematic drawing of synthesis of 3.5-dimethyl-fentanyl analogues

FIG. 10a shows schematic drawing of examples of ‘ring-closed’ analogs of fentanyl

FIG. 10b shows schematic drawing of synthesis of 3.5-dimethyl-fentanyl analogues

FIG. 11a shows schematic drawing of one more example of ‘ring-closed’ analogs of fentanyl-indolylpiperidines

FIG. 11b shows schematic drawing of synthesis of 4-methyl-fentanyl analogue

FIG. 12a shows schematic drawing of synthesis of series of pyridoindoles of benzo[a]-quinolizidine derivatives of fentanyl

FIG. 12b shows schematic drawing of general method for the synthesis of compounds with various aromatic β-substituents

FIG. 13a shows schematic drawing of synthesis of isothiocyanate derivatives of fentanyl

FIG. 13b shows schematic drawings of structures of fentanyl analogues

FIG. 14a shows schematic drawing of replacement of 2-arylethyl substituents at piperidine ring

FIG. 14b shows schematic drawing of synthesis of remifentanil and analogues

FIG. 15a shows schematic drawing of bivalent ligands based on fentanyl

FIG. 15b shows schematic drawing of alternative synthesis of remifentanil

FIG. 16a shows schematic drawings of functionalized fentanyls for the synthesis of bivalent ligands

FIG. 16b shows schematic drawing of alternative synthesis of 4-acyloxymethyl-fentanyls

FIG. 17a shows schematic drawing of newly created bivalent ligands based on fentanyl

FIG. 17b shows schematic drawing of variations in 4-anylino-fragment of fentanyl

FIG. 18a shows schematic drawings of attempts to synthesize fentanyl/adjuvant bivalent compounds

FIG. 18b shows schematic drawing of fluoro-derivative of fentanyl

FIG. 19a shows schematic drawing of another attempt to synthesize fentanyl/δ-agonist bivalent compounds

FIG. 19b shows schematic drawing of replacements of phenyl group of 4-anylino-fragment of fentanyl for heterocycles

FIG. 20a shows schematic drawing of chemical structure of sufentanyl

FIG. 20b shows schematic drawings of structures of some unique fentanyl analogues with opioid antagonist activity

FIG. 21 shows schematic drawing of synthesis of some guanidinium derivatives of fentanyl

FIG. 22 shows schematic drawing of synthesis of 3-methoxy-derivative of fentanyl

FIG. 23 shows schematic drawing of synthesis of 3-carbmethoxy-derivative of fentanyl

FIG. 24 shows schematic drawing of synthesis of -carboxamide derivative of fentanyl

FIG. 25 shows schematic drawing of synthesis of 3,3-dimentyl-fentanyl

FIG. 26 shows schematic drawing of synthesis of some “ring-closed” analogues of fentanyl

FIG. 27 shows schematic drawing of another example of “ring-closed” analogues of fentanyl

FIG. 28 shows schematic drawing of synthesis of ohmefentanyl

FIG. 29 shows schematic drawings of four stereoisomers of ohmefentanyl

FIG. 30 shows schematic drawings of replacement of aniline-moiety for benzylamino group in fentanyl

### DETAILED DISCLOSURE

This invention provides compositions for application by spray nasally or orally in the treatment and/or reversal of substance abuse and addiction, overdose and/or treatment, particularly for fentanyl and fentanyl analogs, of behavioral disorders, such as addiction to opiates and opioids, including heroin, opium, oxycontin, oxycodone, morphine, methodone and tramadol, addiction to prescription drugs such as benzodiazepines, including vallum and xanax, amphetamines, hypnotics, barbitunates, cocaine, methamphetamine, PCP and other addictive and/or behavioral disorders, such as gambling urges and tendencies and sex and pornography addiction, opioid depression, alcohol abuse, self-injurious behaviors, such as self mutilation, kleptomania, and other known other impulse control disorders treatable by agonists and/or antagonists in accordance with the invention.

While opioids and many opioid synthetics, “opiates” such as OxyContin, have presented many addiction and overdose related problems throughout the years, fentanyl and an emerging rash of extremely highly potent fentanyl analogs have been particularly troublesome, with no known overdose antidote, until this inventive method and product. Fentanyl and its analogs have been known for the treatment of severe to moderate pain for several years. As known, fentanyl is many times more potent than morphine for use in chronic pain treatment, especially for the terminally ill. The abuse of this family of drugs is currently an epidemic as new and previously unknown fentanyl analogs have been appearing for street and illicit drug use which are hundreds and even thousands of times more potent than morphine or fentanyl itself, and with no known previous treatment or antidote for overdose and potential deaths.

Paul Janssen, the physician-founder of Janssen Pharmaceutica, now a unit of Johnson & Johnson, first synthesized fentanyl in a Belgian laboratory in 1960. For a time, it was the world's strongest opioid approved for human medical use, soothing excruciating pain especially for the terminally ill and helping to put surgical patients to sleep.

For years, fentanyl-related overdose deaths never exceeded a few thousand, However, an epidemic has taken. hold, with the drugs killing more than 5,000 people in 2014 alone. By September 2017, they accounted for more than 26,000 deaths, half the opioid total. This epidemic has been hastened in the 1990s, when doctors began overprescribing legal painkillers like OxyContin. Abusers eventually turned to their stronger cousin, heroin, which in turn created an opening for dealers to offer fentanyl.

Fentanyl itself is potent almost beyond comprehension. It's prescribed by the millionth of a gram. Two milligrams, the equivalent of seven poppy seeds, can kill. It's often crudely diluted, which makes it difficult for illicit users to determine how much they're consuming.

The drug's rise has coincided with that of the dark web, encrypted messaging apps and cryptocurrencies like Bitcoin, all of which help manufacturers remain anonymous. Hundreds of thousands of doses can be transported via the U.S. Postal Service or FedEx, and slipping past law officers accustomed to tracing narcotics by the truckload.

Fentanyl's astronomical profit margins are thought to have driven its rapid spread and that of its numerous analogs a kilogram from China purchased for $3,800, which, when turned into tablet form, may fetch on the street up to $30 million. Compare that with a kilo of heroin, which wholesales some places for about $50.000 and may generate a profit of just $200.000.

Fentanyl is known as a smuggler's dream, as it is compact, valuable, fantastic for the smugglers and terrible for law enforcement.

There are many other advantages as there is no need to grow vast fields of opium poppies, which must be defended against weather, competitors and government eyes. Raw materials and equipment are cheap. Synthesis takes about a week and requires neither heat nor skills more sophisticated than following a recipe. And in recent years, rogue chemists have unearthed instructions for analogues that researchers discovered decades ago but never put into legitimate use. Sellers offer these variations before governments can outlaw them. Potency and purity vary widely. One dose may produce a euphoric high, while another kills immediately.

In recent times, most fentanyl on America's streets isn't made for pharmaceutical use and then diverted. According to the DEA, it's usually illicitly manufactured in overseas laboratories and more recently in the U.S.A. Mexican cartels have played an increasingly prominent role as well, using networks established for heroin and methamphetamine, but U.S. officials say most of the illicit fentanyl/fentanyl analog trafficking originates in China, one of the world's top manufacturers and exporters of raw pharmaceutical ingredients.

China has a fraught history with opium, dating to when foreign traders imported it in the mid-1800s. Widespread addiction followed, and attempts to stamp out the trade triggered two futile wars against the British.

Today, notwithstanding historical opium abuse, the Chinese consume a fraction of the pharmaceutical opioids Americans do and are thought not to have much of an abuse problem as a result. Yet a strong domestic market for synthetics flourishes, driven by demand abroad. A vast chemical industry operates there with little government oversight, according to the U.S.-China Economic and Security Review Commission, and can legally procure ingredients tightly regulated elsewhere presenting many problems in other countries.

While Chinese authorities are said to at least in part control fentanyl, they have hardly or have not all controlled new analogues, and China didn't begin restricting fentanyl's two most common ingredients until fairly recently, or more than a decade after the U.S. In addition to clandestine operations, according to the DEA, legitimate laboratories routinely manufacture the illicit opioid on the side, and unlike in the U.S., anyone can sell or purchase pill presses, which dealers use to pass off milder drugs, such as OxyContin, when they're actually distributing fentanyl.

Fentanyl was initially developed for parenteral administration. Due to a fast first-pass metabolism, oral administration is not available. A multitude of novel delivery systems and different approaches for fentanyl delivery have been developed in revent decades, including a transdermal system (patch) for long-term treatment of chronic pain (Grape S, Schug S A, Lauer S, Schug B S. Formulations of fentanyl for the management of pain. Drugs. 2010; 70(1):57-72, Nelson L, Schwaner R. Transdermal fentanyl: pharmacology and toxicology. J. Med. Toxicol. 2009; 5(4):230-241); fentanyl buccal tablets, which permit direct absorption of the drug through the oral mucosa providing rapid-onset analgesia for breakthrough pain, has been approved mainly for persistent pain of cancer patients (Jeal W, Benfield P. Transdermal fentanyl: a review of its pharmacological properties and therapeutic efficacy in pain control. Drugs. 1997; 53(1):109-138, Darwish M, Messina J. Clinical pharmacology of buccal tablet for the treatment of breakthrough pain. Exp. Rev. Clin. Pharmacol. 2008; 1(1):39-47, Taylor D R. Fentanyl buccal tablet: rapid relief from breakthrough pain. Exp. Opin. Pharmacother. 2007; 8(17):3043-3051); and oral, transmucosal fentanyl, an intranasal fentanyl spray with a quicker onset (Davis M P. Fentanyl for breakthrough pain: a systematic review. Exp. Rev. Neurotherapeut. 2011; 11(8):1197-1216, Mystakidou K, Panagiotou I, Gouliamos A. Fentanyl nasal spray for the treatment of cancer pain. Exp. Opin. Pharmacother. 2011; 12(10):1653-1659). Thousands of papers are published on fentanyl chemistry and reviewed in (Casy A F, Hassan M M, Simmonds B, Staniforth D. Structure-activity relations in analgesics based on 4-anilinopiperidine. J. Pharm. Pharmacol. 1969; 21(7):434-140, Bagley J R, Kudzma L V, Lalinde N L, et al. Evolution of the 4-anilidopiperidine class of opioid analgesics. Med. Res. Rev. 1991; 11(4):403-436, Vuckovic S, Prostran M, Ivanovic M, et al. Fentanyl analogs: structure-activity relationship study. Curr. Med. Chem. 2009; 16(19):2468-2474, Yadav P, Chauhan J S, Ganesan K, et al. Synthetic methodology and structure activity relationship study of N-[1-(2-phenylethyl)-piperidin-4-yl]-propionamides. Pharm. Sinica. 2010; 1(3):126-139). Its pharmacology reviewed in (Yaksh T L, Noueihed R Y, Durant P A C. Studies of the pharmacology and pathology of intrathecally administered 4-anilinopiperidine analogs and morphine in the rat and cat. Anesthesiol. 1986; 64(1):54-66, Planas E. Fentanyl pharmacological characteristics. Dolor. 2000; 15(1):7-12, Poklis A. Fentanyl: a review for clinical and analytical toxicologist. J. Tox. Clin. Tox. 1995; 33(5):439-447, Andrews C J H, Prys-Roberts C. Fentanyl—a review. Clinics Anaesthesiol. 1983; 1(1):97-122, Mather L E. Clinical pharmacokinetics of fentanyl and its newer derivatives. Clin. Pharmacokinet. 1983; 8(5):422-146, Massey J. Stop the pain: fentanyl is a viable alternative to morphine. JEMS. 2011; 36(8):54-57, Pasero C. Fentanyl for acute pain management. J. Perianesth. Nurs. 2005; 20(4):279-84, Peng P W, Sandler A N. A review of the use of fentanyl analgesia in the management of acute pain in adults. Anesthesiol. 1999; 90(2):576-599, Grass J A. Fentanyl: clinical use as postoperative analgesic-epidural/intrathecal route. J. Pain Symptom Manage. 1992; 7(7):419-430) and history in (Stanley T H. Fentanyl. J. Pain Symptom Manage. 2005; 29(55):S67-S71, Stanley T H. The history and development of the fentanyl series. J. Pain Symptom Manage, 1992; 7(3 Suppl.):S3-S7). Information of fentanyl and its congeners is included in books and reviews on opioid analgesics (Cary A F, Parfitt R T. Opioid Analgesics: Chemistry and Receptors. NY, USA: Springer; 1986, Lenz G R, Evans S M, Walters D E, Hopfinger A J. Opiates. MA, USA: Academic Press; 1986, Lednicer D. Central Analgetics—Chemistry and Pharmacology of Drugs, A Series of Monographs Vol. 1. NY, USA: Wiley-Interscience; 1982, Buschmann H, Christoph T, Friderichs E, Maul C. Analgesics: From Chemistry and Pharmacology to Clinical Application. Weinheim, Germany: Wiley-VCH; 2002, DeStevens G. Analgetics—Medicinal Chemistry, A Series of Monographs, Vol. 5. NY, USA: Academic Press; 1965, Hellerbach J, Schnider O, Besendorf H, Pellmont B. Synthetic Analgesics, Part IIA: Morphinans, Organic Chemistry. Vol. VIII. London, UK: Pergamon Press; 1966, Nathan B, Eddy N B, May E L. Synthetic Analgesics, Part IIB: Benzomorphans—International Series of Monographs on Organic Chemistry Vol. 8. London, UK: Pergamon Press; 1966, Janssen P A J. Synthetic Analgesics, Part I: Diphenylpropylamines—International Series of Monographs on Organic Chemistry Vol. 3. London, UK: Pergamon Press; 1960, Sawynok J, Cowan A. Novel Aspects of Pain Management: Opioids and Beyond. NY, USA: Wiley-Liss; 1999), and also in corresponding chapters of popular textbooks of medicinal chemistry (Lemke T H, Williams D A, Roche V F, Zito S W. Foye's Principles of Medicinal Chemistry. PA, USA: Lippincott Williams and Wilkins, WoltersKluwer Health; 2013, Abraham D J, Rotella D P, Burger's Medicinal Chemistry and Drug Discovery 7th Edition. NJ, USA: John Wiley and Sons, Inc; 2010, Teiggle D J, Taylor J B. Comprehensive Medicinal Chemistry II. London, UK: Elsevier Science; 2006, Thomas G. Fundamentals of Medicinal Chemistry. NJ, USA: Wiley-Blackwell; 2003, Vardanyan R S, Hruby V J. Synthesis of Essential Drugs, Amsterdam. The Netherlands: Elsevier; 2006, Block J, Beale J M. Wilson and Gisvold's Textbook of Organic Medicinal and Pharmaceutical Chemistry 11th Edition. PA, USA: Lippincott Williams and Wilkins; 2003).

Fentanyl, first invented more than 40 years ago, remains the mainstay of anesthesiologists throughout the world.

Fentanyl is well known as an extremely powerful analgesic, 50-100-times more potent than morphine. Its LD₅₀ is of 3.1 mg/kg in rats and 0.03 mg/kg in monkeys. The LD₅₀ in humans is unknown. The safety margin (LD₅₀/lowest ED₅₀) is ˜280 (See Van Bever W F M, Niemegeers C J E, Schellekens K H L, Janssen P A J. N-4-substituted 1-(2-arylethyl)-4-piperidinyl-N-phenylpropanamides, a novel series of extremely potent analgesics with unusually high safety margin. Arzneimittelforschung. 1976; 26(8):1548-1551). Just 0.1 mg of fentanyl is approximately equivalent to 10 mg of morphine and 75 mg of pethidine in analgesic activity. After the subcutaneous administration of 0.1 mg/kg of fentanyl to mice, maximum activity occurred at 10-15 min. The analgesic effect of fentanyl lasts no longer than 30 min after injection. For comparison, the peak activity of morphine occurred at 45 min after injection of 20 mg/kg of the. The duration of action at this dosage level of morphine appears to be greater than 1 h. The ED₅₀ and 95% limits for fentanyl citrate injections is calculated to be 0.08 (0.045-0.142) mg/kg, while for morphine it is 15 (12-20) mg/kg (Gardocki J F, Yelnosky J. Some of the pharmacologic actions of fentanyl citrate and droperidol. Toxicol. Appl. Pharmacol. 1964; 6(1):48-62). Data on the relative analgesic potency of fentanyl and other analgesics on humans have been discussed (Chrubasik J, Wust H, Schulte-Monting J, et al. Relative analgesic potency of epidural fentanyl, alfentanil, and morphine in treatment of postoperative pain. Anestheziol. 1988; 68(6):929-933, Reynolds L, Rauck R, Webster L, et al. Relative analgesic potency of fentanyl and sufentanil during intermediate-term infusions in patients after long-term opioid treatment for chronic pain. Pain. 2004; 110(1-2):182-188). Detailed fentanyl pharmacology is described in a few reviews (Planas E. Fentanyl pharmacological characteristics. Dolor. 2000; 15(1):7-12, Barutell C, Ribera M V, Martinez P, et al. Fentanyl. Dolor. 2004; 19(2):98-104, Andrews C J H, Prys-Roberts C. Fentanyl—a review. Clin. Anaesthesiol. 1983; 1(1):97-122). The action of fentanyl is qualitatively similar to morphine. Cortical depression is minimal. Respiratory alterations may last longer than the analgesic effect. No significant cardiovascular effects were observed at usual therapeutic doses.

Fentanyl rapidly distributes with sequestration in fat and it extensively binds to human plasma proteins. It is metabolized mainly by the liver and is excreted via the kidney. Elimination half-life varies from 6 to 32 h. Action starts almost immediately with intravenous administration and after 7-8 min with intramuscular dosing. The peak effect that the drug achieves is observed in 5-15 min following intravenous injection. Duration of the analgesic effect is 1-2 h on intramuscular administration. So it has a faster onset of action but a shorter duration of action than morphine.

Fentanyl is also known to act addictively with other opioids and depressants. Tolerance may be developed with prolonged use and correspondingly minimal effective dose may be increased. Physical dependence may develop over a few days. Possible adverse effects have been described as respiratory depression, bradycardia, nausea and vomiting, and some muscle, especially chest wall rigidity ((Planas E. Fentanyl pharmacological characteristics. Dolor. 2000; 15(1):7-12, Barutell C, Ribera M V. Martinez P, et al. Fentanyl. Dolor. 2004; 19(2):98-104, Andrews C J H, Prys-Roberts C. Fentanyl—a review. Clin. Anaesthesiol. 1983; 1(1):97-122)).

Fentanyl acts preferentially on μ receptors (Schulz R, Wuster M, Rubini P, Herz A. Functional opiate receptors in the guinea-pig ileum: their differentiation by means of selective tolerance development. J. Pharmacol. Exp. Ther. 1981; 219:547-550, Mohamed Y H, Essawi M Y H, Portoghese P S. Synthesis and evaluation of 1- and 2-substituted fentanyl analogs for opioid activity. J. Med. Chem. 1983; 26(3):348-352, Magnan J, Paterson S J, Tavani A, Kosterlitz W. The binding spectrum of narcotic analgesic drugs with different agonist and antagonist properties. Arch. Pharmacol. 1982; 319(3):197-205, Maguire P, Tsai N, Kamal J, et al. Pharmacological profiles of fentanyl analogs at μ, δ and κ opiate receptors. Eur. J. Pharmacol. 1992; 213(2):219-225, Jin W Q, Paterson S J, Kosterlitz H W, Casy A F. Interactions of some 4-anilino-piperidines and 4-phenylpiperidines with the μ-, δ- and κ-binding sites. Life Sci. 1983; 33(Suppl. 1):251-253, Boas R A, Villiger J W. Clinical actions of fentanyl and buprenorphine. The significance of receptor binding. Brit. J. Anaesth. 1985; 57(2):192-196). The opioid activity of fentanyl was evaluated according to comprehensive protocols which showed that it is more potent than morphine in the guinea pig ileum (GPI) and mouse vas deferens (MVD) assays. In the GPI, it has an IC₅₀ of 3.45±0.45×10⁻⁹ M compared with 3.31±0.94×10⁻⁸ M for morphine. The IC₅₀ of fentanyl in the MVD test is 9.45±4.05×10⁻⁹ M while that for morphine was 1.94±0.34×10⁻⁷ M (Mohamed Y H, Essawi M Y H, Portoghese P S. Synthesis and evaluation of 1- and 2-substituted fentanyl analogs for opioid activity. J. Med. Chem. 1983; 26(3):348-352). The literature data show that all of fentanyl profile compounds of the 4-anilidopiperidin series are highly μ-selective, but could also produce affinity for δ- and κ-opiate receptors. Moreover, it seems that as their μ affinity increases, their selectivity decreases (Maguire P, Tsai N, Kamal J, et al. Pharmacological profiles of fentanyl analogs at μ, δ and κ opiate receptors. Eur. J. Pharmacol. 1992; 213(2):219-225). In general, all the compounds of the series tested had high affinity for the μ-binding site, low affinity for the δ-site and were almost inactive at the κ-site (Jin W Q, Paterson S J, Kosterlitz H W, Casy A F. Interactions of some 4-anilino-piperidines and 4-phenylpiperidines with the μ-, δ- and κ-binding sites. Life Sci. 1983; 33(Suppl. 1):251-253, Boas R A, Villiger J W. Clinical actions of fentanyl and buprenorphine. The significance of receptor binding. Brit. J. Anaesth. 1985; 57(2):192-196).

Biotransformation of fentanyl was most pronounced in the liver (Lehmann K A, Weski C, Hunger L. Biotransformation of fentanyl. II. Acute drug interactions in rats and men. Anaesthesist. 1982; 31(5):221-227). The main degradation pathway in vivo is oxidative dealkylation leading to the formation of the main metabolites, phenylacetic acid and norfentanyl (Lehmann K A, Hunger L, Brandt K, Daub D. Biotransformation of fentanyl. III. Effect of chronic drug administration on distribution, metabolism and excretion in the rat. Anaesthesist. 1983; 32(4):165-173).

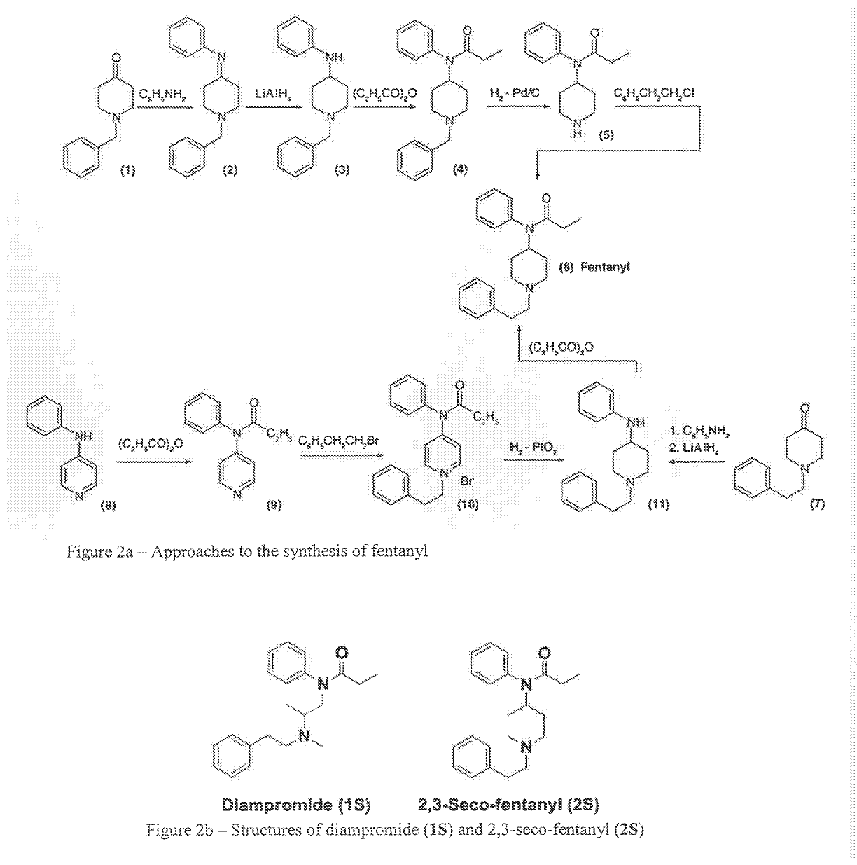

The first synthesis of fentanyl (6) was proposed by P A J Janssen, starting with 1-benzypiperidin-4-one (1), which was condensed with aniline to give the corresponding Schiff base (2) (FIG. 2a). The double bond in the obtained imine (2) was reduced with lithium aluminum hydride, and the resulting 1-benzyl-4-anilinopiperidine (3) was acylated using propionic anhydride. The resulting 1-benzyl-4-N-propinoyl-anilinopiperidine (4) underwent debenzylation, using standard H₂—Pd/C conditions, to give 4-N-propanoylanilinopiperidine or norfentanyl (5), which was then N-alkylated by 2-phenylethylchloride (or tosylate) to give the desired fentanyl (6) (Janssen P A J, Gardocki J F. U.S. Pat. No. 3,141,823. 1964, Janssen P A J. FR M2430. 1964, Janssen P A, Niemegeers C J, Dony J G. The inhibitory effect of fentanyl and other morphine-like analgesics on the warm water induced tail withdrawl reflex in rats. Arzneimittelforschung. 1963; 13:502-507, Janssen P A J, U.S. Pat. No. 3,164,600. 1965, Janssen P A J. The development of new synthetic narcotics. In: Estafanous F G, editor. Opioids in Anesthesia II. MA, USA: Butterworth-Heinemann; 1983. pp. 37-44).

Later, a shorter modified synthesis by the same scheme was proposed, starting directly from 1-(2-phenethyl)piperidin-4-one (7) (FIG. 2a) (Jonczyk A, Jawdosiuk M, Makosza M, Czyzewski J. Search for a new method for synthesis of the analgesic agent “Fentanyl” Przeml. Chem. 1978; 57(3):131-134). Another approach, which included a step of hydrogenation of pyridinium salt (10) has been developed (Zee S-H, Wang W-K. A new process for the synthesis of fentanyl. J. Chin. Chem. Soc. 1980; 27(4):147-449). Fentanyl (6) was prepared starting from 4-anilinopyridine (8), which on propionylation gave 4-N-propinoylanilidopyridine (9). The last was alkylated with 2-phenylethylbromide to give pyridinium salt (10), hydrogenation of which over PtO₂ gave known 4-N-anilinopiperidine derivative (11), which was propionlated to the desired fentanyl (6) (FIG. 2a) (Zee S-H, Wang W-K. A new process for the synthesis of fentanyl. J. Chin. Chem. Soc. 1980; 27(4):147-149).

The analgesic potency of fentanyl is approximately 300-times higher than that of morphine in the tail withdrawal test in rats. It has been enhanced up to 10,000-times with that of the fentanyl analogs, such as (carfentanil; 45) compared to that of morphine by making a series of minor, but sensitive changes to the fentanyl structure.

Typical fentanyl derivatives include, for example, the replacement of piperidine ring for pyrrolidine or azepine rings, as well as synthesis of open chain compounds; replacement of phenyl group in the phenethyl—part of molecule for some aromatic heterocyles, mainly for thiophene and tetrazole; insertion of carbomethoxy—or methoxymethyl—into the forth position of piperidine ring; changes had been done in the 4-anilino-part of fentanyl molecule, mainly via replacement of hydrogen atoms in aromatic ring for fluorine atoms, or replacement of whole benzene ring for an aromatic heterocycle; additional methyl groups have been inserted into different positions of the piperidine ring; replacement of propionyl-group in the 4-anilido-fragment for several other acyl groups, which gave a novel series of extremely potent analgesics.

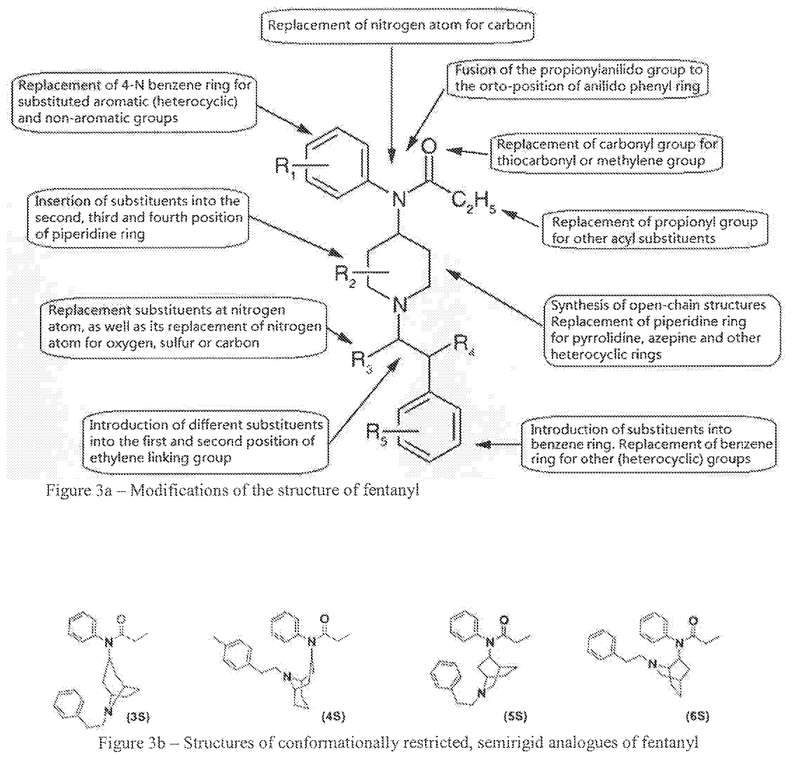

Several possible synthetic routes for the creation of a series of novel potent analgesics have been reported starting with the fentanyl core structure to produce a series of powerful fentanyl analogs (See FIG. 3a).

Numerous fentanyl analogs have been synthesized since 1964, including acyclic open chain compounds which have been shown to be strong analgesics (Wright W B, Jr, Brabander H J, Hardy R A., Jr Synthetic analgesics. III. Basic anilides and carbanilates containing the phenylalkyl moiety. J. Org. Chem. 1961; 26:485-490). One of them, diampromide (1S) which produces effects similar to other opioid analgesics, and is around the same potency as morphine with an ED₅₀ of 4 mg/kg (Casy A F, Hassan M M A. Analgesically active basic anilides: stereospecificity and structure of the basic group. J. Pharm. Pharmacol. 1967; 19(1):17-24, Wright W B, Jr, Hardy R A., Jr Synthetic analgesics. IV. Synthesis of enantiomers of basic anilides containing the phenalkyl moiety. J. Med. Chem. 1963; 6:128-130), another is 2,3-seco-fentanyl (2S) whose central-analgesic activity was found to be 40-times lower than fentanyl, but still 5-6-times higher than that of morphine (FIG. 2b) (Ivanovic M D, Micovic I V, Vuckovic S, et al. The synthesis and pharmacological evaluation of (±)-2,3-seco-fentanyl analogs. J. Serb. Chem. Soc. 2004; 69(11):955-968).

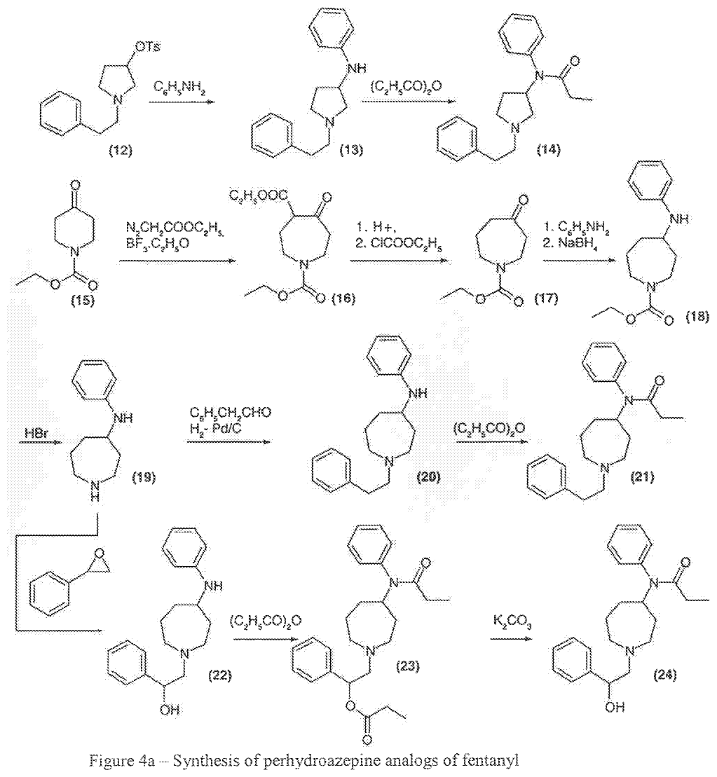

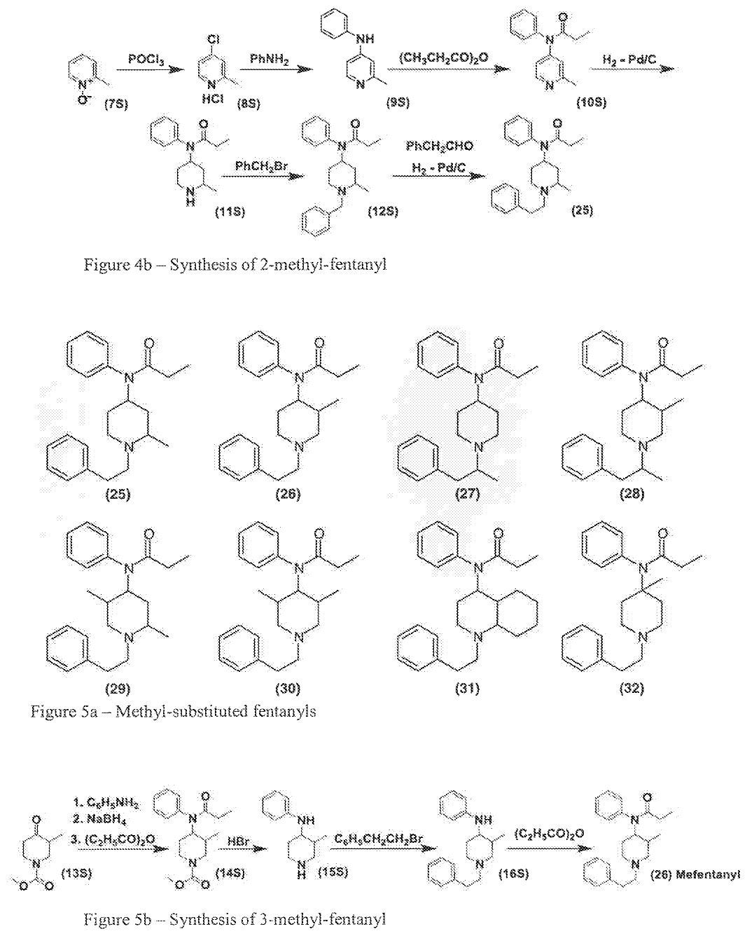

The preparation and analgesic properties of a series of fentanyl analogs with piperidine ring contraction or expansion, replacing it for pyrrolidine (14) or perhydroazepines (21) was described. The compound (14) was prepared by the nucleophilic replacement of the tosyl- or promo-groups in compounds such as 3-tosyl-1-phenethylpyrrolidine (12) with aniline to get 3-anilinopyrrolidine derivative (13). Further acylation with different acid chlorides gave compounds with significant analgesic activity. The most active compounds were those in which the pyrrolidine N-substituent was phenethyl, O-methoxy-phenoxyethyl, or benzoylethyl. Compound 14 was found to be the most active with ED₅₀ 2 mg/kg (FIG. 4a) (Helsley G C, Lunsford C D, Welstead W J, Jr, et al. Synthesis and analgetic activity of some 1-substituted 3-pyrrolidinylanilides and dihydrobenz-oxazinones. J. Med. Chem. 1969; 12(4):583-586).

The synthesis and analgesic properties of 4-(propananilido)perhydroazepines (21) also was described (FIG. 4a) (Finney Z G, Riley T N. 4-Anilidopiperidine analgesics. 3. 1-Substituted 4-(propananilido)-perhydroazepines as ring expanded. analogs. J. Med. Chem. 1980; 23(8):895-899, DeRuiter J, Andurkar S, Riley T N, et al. Investigation of the synthesis and analgesic activity of 1-substituted 4-(propananilido) perhydroazepines. J. Het. Chem. 1992; 29(4):779-786). The desired perhydroazepine derivative (21) was prepared via ring homologation of 1-carbethoxy-4-piperidinone (15) with ethyl diazoacetate and boron trifluoride to provide the β-keto ester (16), which after hydrolysis and decarboxylation in refluxing HCl, followed by carbamoylation with ethyl chloroformate, yielded perhydroazepinone (17) (FIG. 4a). Condensation of the obtained (17) with aniline, followed by NaBH₄ reduction, provided 1-carbethoxy-4-anilinoperhydroazepine (18), which was decarbamoylated in refluxing 48% HBr to give (19). The appropriate 1-phenethyl substituent was incorporated by reductive alkylation of the first position of the perhydroazepine using as a carbonyl component phenylacetaldehyde to give 1-substituted 4-anilinoperhydroazepine derivative (20). The target compound (21) was obtained by propionylation of the last.

The 1-methyl, 1-benzyl, as well as 1-allyl and 1-(cyclopropylmethyl) derivatives also were prepared via direct alkylation of 19. Compound 21 was the most active among this series with ED₅₀ 2 mg/kg (Finney Z G, Riley T N. 4-Anilidopiperidine analgesics. 3. 1-Substituted 4-(propananilido)-perhydroazepines as ring expanded analogs. J. Med. Chem. 1980; 23(8):895-899, DeRuiter J, Andurkar S, Riley T N, et al. Investigation of the synthesis and analgesic activity of 1-substituted 4-(propananilido) perhydroazepines. J. Het. Chem. 1992; 29(4):779-786).

Either piperidine ring expansion (21) or ring contraction (14) of fentanyl significantly decreases analgesic activity of obtained entities. The most active among synthesized compounds were 14 and 21, which are N-phenethyl entities with 150- to 200-fold less potency than the corresponding piperidine homolog fentanyl (6).

Treatment of 19 with styrene oxide and following the transformations 22→24 afforded the new derivative 24, which, in the tail-flick assay had greater analgesic activity than other reported members of this series (FIG. 4a) (DeRuiter J, Andurkar S, Riley T N, et al. Investigation of the synthesis and analgesic activity of 1-substituted 4-(propananilido) perhydroazepines. J. Het. Chem. 1992; 29(4):779-786).

A series of conformationally restricted, semirigid analogs of fentanyl as N-substituted propionylananilidonortropanes (38) (Bagley J R, Riley T N. Synthesis and conformational analysis of isomeric 3-propananilidotropanes. J. Het. Chem. 1977; 14(4):599-602, Riley T N, Bagley Jr, 4-Anilidopiperidine analgesics. 2. A study of the conformational aspects of the analgesic activity of the 4-anilidopiperidines utilizing isomeric N-substituted 3-(propananilido)nor-tropane analogs, J. Med. Chem. 1979; 22(10):1167-1171), nor-granatane (4S) (Fernandez M J, Huertas R M, Galvez E, et al. Synthesis, and structural, conformational and pharmacological studies of new fentanyl derivatives of the norgranatane system. J. Chem. Soc. Perkin Trans. 1992; 24:687-695), azabicyclo[2.2.2]octanes (5S) and (6S) (Law S-J, Lewis D H, Borne R F. Synthesis and stereochemical analysis of isomeric N-substituted 5- and 6-propanilido-2-azabicyclo[2.2.2]octanes, J. Het. Chem. 1978; 15(2):273-280, Borne R F, Law S-J, Kapeghian J C, Masten L W. Evaluation of 2-azabicyclo[2.2.2]octane analogs of 4-anilidopiperidine analgesics. J. Pharm. Sci. 1980; 69(9):1104-1106) were synthesized, and stereochemically characterized. The scheme of synthesis of these compounds is practically the same as described above and has been started with appropriate ketones.

The 3-β-(chair conformation) of 3S displayed greater analgesic, activity (ED₅₀ 0.047 mg/kg) in mice than the respective 3-a-isomer (Bagley J R, Riley T N. Synthesis and conformational analysis of isomeric 3-propananilidotropanes. J. Het. Chem. 1977; 14(4);599-602). 9-phenethyl-3-α-(N-tolylamido)norgranatane (4S) showed an ED₅₀ value of 100 mg/kg (Fernandez M J, Huertas R M, Galvez E, et al. Synthesis, and structural, conformational and pharmacological studies of new fentanyl derivatives of the norgranatane system. J. Chem. Soc. Perkin Trans. 1992; 24:687-695). The most potent fentanyl analogs of the isomeric azabicyclo[2.2.2]octane derivatives (5S, 6S) pair had a potency 600-times less than that of fentanyl (FIG. 3b) (Law S-J, Lewis D H, Borne R F. Synthesis and stereochemical analysis of isomeric N-substituted 5- and 6-propanilido-2-azabicyclo[2.2.2]octanes. J. Het. Chem. 1978; 15(2):273-280, Borne R F, Law S-J, Kapeghian J C, Masten L W. Evaluation of 2-azabicyclo[2.2.2]octane analogs of 4-anilidopiperidine analgeics. J. Pharm. Sci. 1980; 69(9):1104-1106).

### Insertion of Methyl Substituent in Different Positions of the Fentanyl Structure

The next series of changes in fentanyl structure involves insertion of different substituents into the fentanyl structure. And these substituents could be either simple as alkyl or aryl groups, or functional groups.

To check the effect of methyl substitution on the piperidine ring on analgesic activity of the 2-methyl-, 3-methyl-, 4-methyl-, 2,5-dimethyl and 3,5-dimethyl-fentanyl derivatives were prepared (FIG. 5a).

2-methyl-fentanyl (25) possessing analgesic activity in the rat with an ED₅₀ of 0.665 mg/kg (Riley T N, Hale D B, Wilson M C. 4-Anilidopiperidine analgesics. I. Synthesis and analgesic activity of certain ring methylated-1-substituted 4-propananilidopiperidines. J. Pharmaceut. Sci. 1973; 62(6):983-986) was proposed to be prepared from 2-methylpyridine N-oxide (7S). On interaction with phosphorous oxychloride (7S) was transformed to 4-chloro-2-methylpyridine hydrochoride (8S), which on heating with aniline gave 4-anilino-2-methylpyridine (9S). Then (9S) was acylated by propionic anhydride to give compound (10S). Hydrogenation of (10S) using Pd/C catalyst yielded 2-methyl-4-(N-phenylpropanamido)piperidine (11S), which after benzylation gave product (12S), which on reductive amination in the presence of phenylacetaldehyde gave the desired (25), possessing analgesic activity in the rat with an ED50 of 0.665 mg/kg. (Scheme 6 in Riley T N, Hale D B, Wilson M C. 4-Anilidopiperidine analgesics. I. Synthesis and analgesic activity of certain ring methylated-1-substituted 4-propananilidopiperidines. J. Pharmaceut. Sci. 1973; 62(6):983-986) The question of why authors avoided direct alkylation with reagents like phenylethylbromide remains open. This synthesis of 3-methyl-fentanyl (26) using the same approach was patented in Riley T N, Hale D B. U.S. Pat. No. 3,923,992. 1975 (FIG. 4b). The synthesis of 3-methyl-fentanyl (26) using the same approach was patented (Riley T N, Hale D B. U.S. Pat. No. 3,923,992. 1975). In the same paper (Riley T N, Hale D B. Wilson M C. 4-Anilidopiperidine analgesics. I. Synthesis and analgesic activity of certain ring methylated-1-substituted 4-propananilidopiperidines. J. Pharmaceut. Sci. 1973; 62(6):983-986), the preparation of 3-methyl-(26) and 2.5-dimethyl-(29) fentanyl derivatives using the same synthetic route is described, and ED₅₀ values of 0.04 and 0.803 mg/kg, respectively were reported. Obtained data indicate that only 3-methylation has a major effect for enhancing analgesic potency, whereas 2-methyl or 2,5-dimethyl substitution is detrimental to analgesic activity. However, comparison with data obtained by other groups suggested this data may not be correct (Vartanyan S A, Vartanyan R S, Zhamagortsyan V N, et al. SU736583. 1985, Vartanian R S, Airapetyan G K, Markaryan E A, et al. SU1100848. 1984, Vartanyan R S, Martirosyan V O, Vartanyan S A, et al. Synthesis and analgesic activity of 4-anilides of 1-substituted 2,5-dimethylpiperidines. Khim. Farm. Zh. 1989; 23(5):562-565, Karapetyan A A, Struchkov YuT, Timofeeva T V, et al. Structure and activity of phenaridine stereoisomers. Khim. Farm. Zh. 1989; 23(5):565-572, Vartanyan R S, Martirosyan V O, Vartanyan S A, et al. Stereochemistry and biological properties of the new narcotic analgesic phenaridine. Khim. Farm. Zh. 1989; 23(5):573-578).

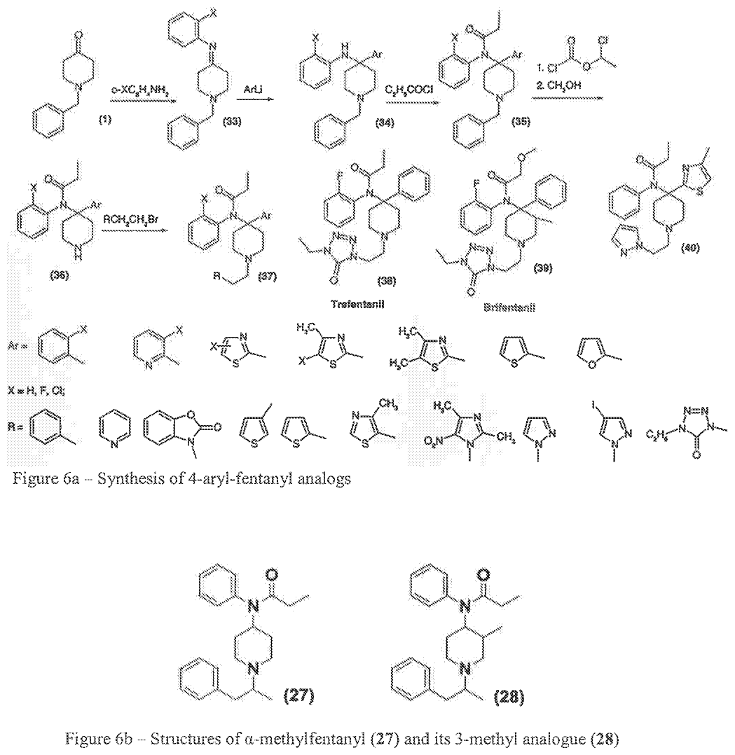

This article (Riley T N, Hale D B, Wilson M C. 4-Anilidopiperidine analgesics. I. Synthesis and analgesic activity of certain ring methylated-1-substituted 4-propananilidopiperidines. J. Pharmaceut. Sci. 1973; 62(6):983-986) was followed with another with a different approach to the synthesis of 3-methylfentanyl derivatives (Van Bever W F M, Niemegeers C J E, Janssen P A J. Synthetic analgesics. Synthesis and pharmacology of the diastereoisomers of N-[3-methyl-1-(2-phenylethyl)-4-piperidyl]-N-phenylpropanamide and N-[3-methyl-1-(1-methyl-2-phenylethyl)-4-piperidyl]-N-phenylpropanamide. J. Med. Chem. 1974; 17(10):1047-1051). This method (FIG. 5b) started with 3-methyl-4-oxopiperidinecarboxylate (13S) which was condensed with aniline and the obtained imine was reduced to the amine, which were propionylated to afford a mixture of cis-(±) and trans-(±) methyl 3-methyl-4-[N-(1-propionoxy)-N-phenylamino]-1-piperidinecarboxylates (14S), which were separated by fractional crystallization (isomers with equatorial 3-Me group were defined as trans-, with axial 3-Me cis-). The N-carbalkoxy groups of the obtained cis-(±) and trans-(±) isomers were removed by brief refluxing in 48% HBr giving the corresponding cis-(±) and trans-(±) diastereoisomers (15S), which were separated to enantiomers by fractional crystallization of their tartaric salts. The D-tartaric acid salt of cis-(±)-(15S) was converted to free base to give optically pure cis-(−) (15S). Similarly, the L-tartaric acid salt of cis-(±)-(15S) afforded the corresponding cis-(+) (15S). Alkylation of the obtained 3-methylpiperidineamines (15S) with 2-phenylethyl bromide yielded the corresponding 3-methyl-1-(2-phenylethyl)-N-phenyl-4-piperidineamines respectively cis-(±)-(16S), cis-(−)-(16S), cis-(+)-(16S), and trans-(±)-(16S). Treatment of the obtained isomers of (16) with propionic anhydride afforded the end products cis-(±)-(26), trans-(±)-(26), cis-(−)-(26) and cis-(+)-(26). The structure assignments were confirmed by NMR spectroscopy using differences in the splitting pattern of the 4-proton of the piperidine ring, and assuming a chair conformation for the piperidine ring, the most predominant conformer would have an equatorial 4-N-phenylpropanamide group. The cis-(+)-N-(3-methyl-1-(2-phenylethyl)-4-piperidyl]-N-phenylpropan-amide (26) is an extremely potent analgesic agent with an ED₅₀ 0.00058 which is up to 6700 times higher than that of morphine. It has a fast onset of action, a shorter duration of action, and a high safety margin. A general conclusion is that the trans-compound (26) is more potent than fentanyl, while the corresponding cis-diastereoisomer (26) is approximately eight times more active than fentanyl. The analgesic activity of (26) as expected is mainly due to one enantiomer, namely the cis-(+) compound (26), which is about 16 times more potent than fentanyl, while its cis-(−) counterpart is 120 times less potent than the cis-(+). It was of interest to know the absolute configuration of product with maximal activity. The absolute configuration of the isomers are: cis-(+)-(26) as (3R,4S), cis-(−)-26) as (3S,4R), trans-(+)-(26) as (3S,4S), trans-(−)-(26) as (3R,4R) (Vartanyan S A, Vartanyan R S, Zhamagortsyan V N, et al. SU736583. 1985). Isomeric α-methyl fentanyl (27), obtained by the same synthetic method but containing an additional methyl group in the side chain in the α position to the basic nitrogen, also displayed high activity (ED₅₀ 0.0085 mg/kg) close to that of fentanyl (ED₅₀ 0.011 mg/kg). Insertion of two methyl groups simultaneously (28) in this position led to enhancement of activity depending on orientation of the 3-methyl substitution in the compounds with ED₅₀'s from 0.011 for the (+)-cis, to 0.00075 mg/kg for the (−)-cis-isomers (Vartanyan S A, Vartanyan R S, Zhamagortsyan V N, et al. SU736583. 1985). (FIG. 6b)

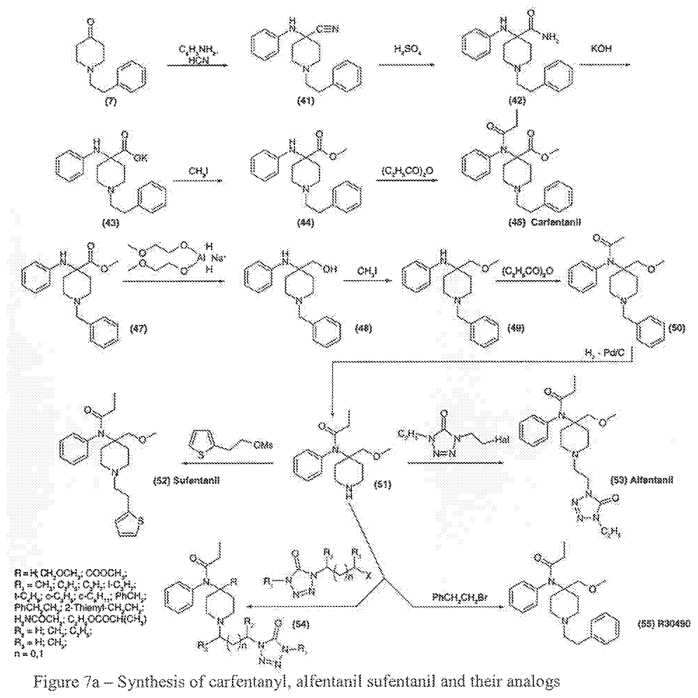

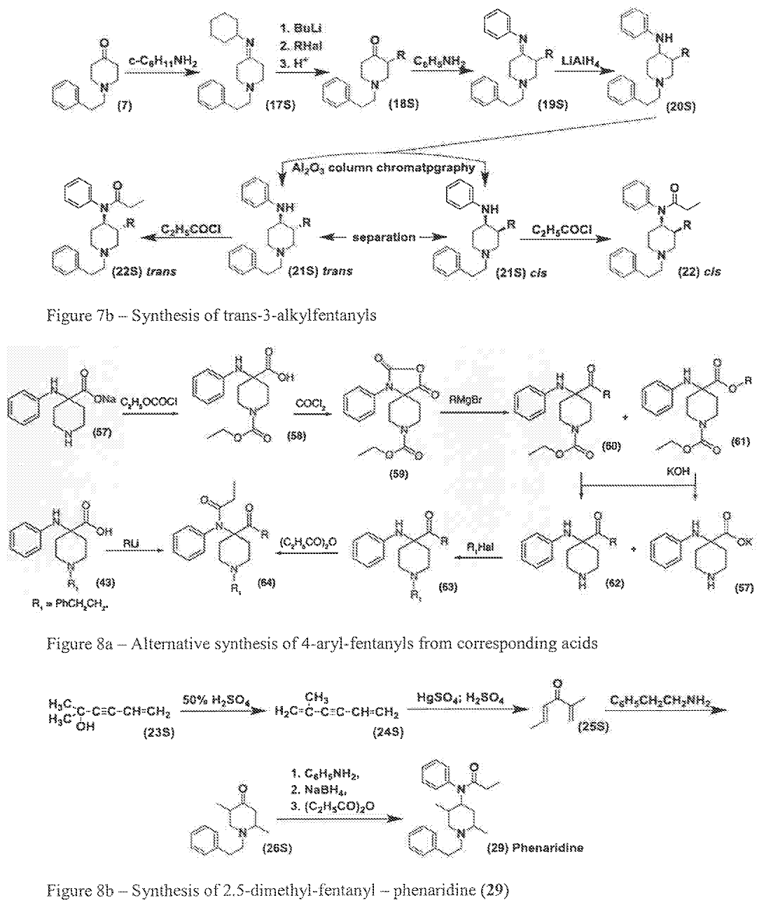

Another method for the synthesis of cis- and trans-3-alkylfentanyl analogs was developed (Vartanian R S, Airapetyan G K, Markaryan E A, et al. SU1100848. 1984) (FIG. 7b) In this case the N-phenethyl-4-piperidone (7) was converted to the cyclohexylimine derivative (17S), which was α-deprotonated with butyllithium and the resulting imine anion was alkylated with alkyl halides to give a variety of piperidin-4-ones (18S), which were reacted with aniline to form the corresponding Schiff bases (19S). Reduction of (19S) with LiAlH₄ yielded mixtures of cis-/trans-3-alkyl-4-anilinopiperidines (20S). The diastereoisomers were separated by column chromatography to yield the pure cis- and trans-isomers. Finally, N-propionylation of the obtained amines afforded twelve new fentanyl analogs (22S). Except for the known (±)-cis-3-methylfentanyl and the novel (±)-cis-3-ethylfentanyl, the others were inactive or less active than fentanyl itself.

The cis-(+)-N-(3-methyl-1-(2-phenylethyl)-4-piperidyl]-N-phenylpropan-amide (26) is an extremely potent analgesic agent with an ED₅₀ value of 0.00058 mg/kg, which is up to 6700-times higher than that of morphine. It has a fast onset of action, a shorter duration of action, and a high safety margin. Its cis-(−) counterpart is 120-times less potent than the cis-(+).

Isomeric α-methylfentanyl (27), obtained by the same synthetic method and containing an additional methyl group in the side chain in the a position to the basic nitrogen, also displayed high activity (ED₅₀=0.0085 mg/kg) close to that of fentanyl (ED₅₀=0.011 mg/kg). Insertion of two methyl groups simultaneously (28) in this position led to enhancement of activity depending on orientation of the 3-methyl substitution in the compounds with ED₅₀ values from 0.011 for the (+)-cis, to 0.00075 mg/kg for the (−)-cis-isomers (Van Bever W F M, Niemegeers C J E, Janssen P A J. Synthetic analgesics. Synthesis and pharmacology of the diastereoisomers of N-[3-methyl-1-(2-phenylethyl)-4-piperidyl]-N-phenylpropanamide and N-[3-methyl-1-(1-methyl-2-phenylethyl)-4-piperidyl]-N-phenylpropanamide. J. Med. Chem. 1974; 17(10):1047-1051).

Another method for the synthesis of cis- and trans-3-alkylfentanyl analogs via alkylation of cyclohexylimine derivative of 1-(2-phenethyl) piperidin-4-ones was developed (FIG. 7b) (Ivanovic M D, Micovic I V, Vuckovic S, et al. The synthesis and preliminary pharmacological evaluation of the racemic cis and trans 3-alkylfentanyl analogs. J. Serb. Chem. Soc. 2004; 69(7):511-526). Except for the known (±)-cis-3-methylfentanyl and the novel (±)-cis-3-ethylfentanyl, the others were inactive or less active than fentanyl itself. The synthesis of 2.5-dimethyl-fentanyl-phenaridine (29)—started with 2-methylhex-5-en-3-yn-2-ol (23S), which underwent dehydration forming dienyne (24S). Triple bond hydration and simultaneous rearrangement of the intermediate allyl-compound gave dienone (25S), which via interaction with phenethylamine was cyclized to desired piperidinone-4 (26S). It was converted to the 2.5-dimethyl-fentanyl by the known protocol via consequential reactions with aniline, followed by hydrogenation to amine and, finally propionylation to give desired (29) as a mixture of isomers (Vartanyan R S, Martirosyan V O, Vartanyan S A, et al. Synthesis and analgesic activity of 4-anilides of 1-substituted 2,5-dimethylpiperidines. Khim. Farm. Zh. 1989; 23(5):562-565, Karapetyan A A, Struchkov YuT, Timofeeva T V, et al. Structure and activity of phenaridine stereoisomers. Khim. Farm. Zh. 1989; 23(5):565-572). (FIG. 8b)

Phenaridine as a mixture of isomers is slightly superior to fentanyl in potency and duration of action. Three isomers have been separated chromatographically and investigated by NMR spectroscopy which revealed that the isomers differ in orientation of the methyl groups in the piperidine ring. In the first one (45% content of the obtained isomeric mixture) the methyl groups oriented as 2-equatorial-5-axial, in the second (40% content) both are equatorial, and in the third one (15%), both methyl groups are oriented axial (Vartanyan R S, Martirosyan V O, Vartanyan S A, et al. Synthesis and analgesic activity of 4-anilides of 1-substituted 2,5-dimethylpiperidines. Khim. Farm. Zh. 1989; 23(5):562-565, Karapetyan A A, Struchkov YuT, Timofeeva T V, et al. Structure and activity of phenaridine stereoisomers. Khim. Farm. Zh. 1989; 23(5):565-572, Vartanyan R S, Martirosyan V O, Vartanyan S A, et al. Stereochemistry and biological properties of the new narcotic analgesic phenaridine. Khim. Farm. Zh. 1989; 23(5):573-578). Absolute configuration of isomers by x-ray investigations was determined (Karapetyan H A, Struchkov Yu T, Martirosyan V O, et al. The structure of analgesics of the 4-anilinopiperidine series. III. Structure of 1(e)-(2-phenethyl)-2(e),5(e)-dimethyl-4(e)-(N-propionylanilino) piperidine bisulfate. Zh. Strukt. Kh. 1990; 31(2):141-145, Karapetyan A A, Timofeyeva T V, Struchkov YuT, Martirosyan V O. Conformation of the 4-(propionylanilino) pharmacophore in stereoisomers of the 2,5-dimethyl derivative of phentanyl (phenaridine) Khim. Farm. Zh. 1992; 26(9-10):25-28). The duration of analgesic effects of the separated isomers was 125, 105, and 165 min for the respective isomers. The overall analgesic activity was comparable to that of fentanyl. Phenaridine itself (mixture of isomers) has an ED₅₀ value of 0.0048 mg/kg (rats, subcutaneous, tail flick test; duration of action 35 min) (Vartanyan R S, Martirosyan V O, Vartanyan S A, et al. Stereochemistry and biological properties of the new narcotic analgesic phenaridine. Khim. Farm. Zh. 1989; 23(5):573-578).

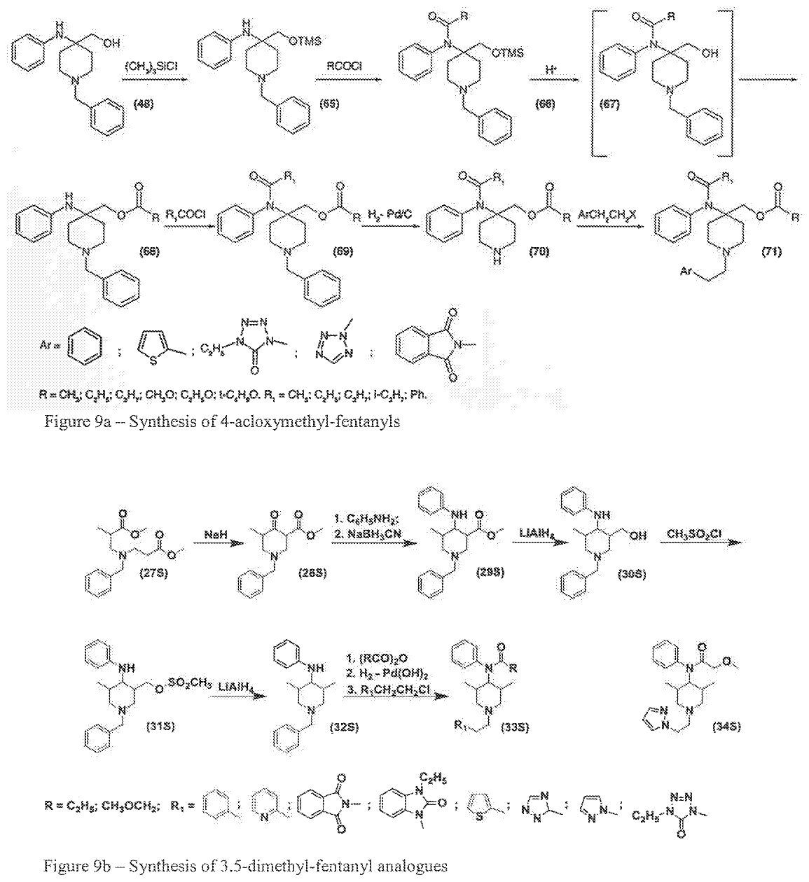

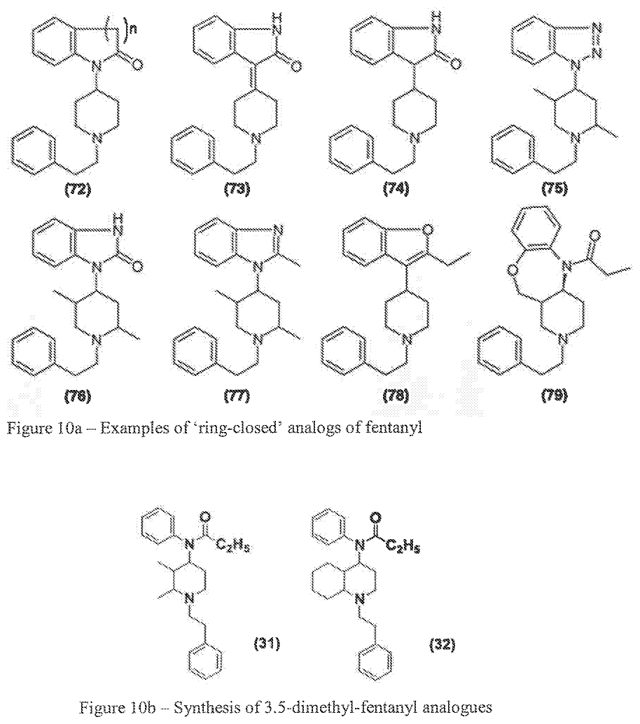

An original method of synthesis was proposed for the synthesis of 3.5-dimethyl-fentanyl analogs. The compounds obtained as diastereomers, were tested for analgesic activity on mouse hot plate test and the majority of the compounds displayed good activity. The most promising was compound 3.5-dimethyl-fentanyl with an ED₅₀ value of 0.0025 mg/kg (FIG. 9b) (Karapetyan H A, Struchkov Yu T, Martirosyan V O, et al. The structure of analgesics of the 4-anilinopiperidine series. III. Structure of 1(e)-(2-phenethyl)-2(e),5(e)-dimethyl-4(e)-(N-propionylanilino) piperidine bisulfate. Zh. Strukt. Kh. 1990; 31(2):141-145, Karapetyan A A, Timofeyeva T V, Struchkov YuT, Martirosyan V O. Conformation of the 4-(propionylanilino) pharmacophore in stereoisomers of the 2,5-dimethyl derivative of phentanyl (phenaridine) Khim. Farm. Zh. 1992; 26(9-10):25-28, Dzhingozyan V K, Martirosyan V O, Karapetyan A A, et al. Effect of 2-methyl substituent on the conformation of 1-(2-phenylethyl) pharmacophore in 4-propanoyl(phenyl)piperidines. Khim. Farm. Zh. 1996; 30(8):40-42, Lalinde N L, Moliterni J, Spencer UK. U.S. Pat. No. 4,939,161, 1990). 2.3-dimethyl-fentanyl (31) is not described in the literature. Decahydroquinoline analog (32) and other derivatives of the same series were synthesized starting from the 4-oxodecahydroquinoline showed low analgesic activities (ED₅₀=25-50 mg/kg) (FIG. 10b) (Prost M. DE2656678. 1977).

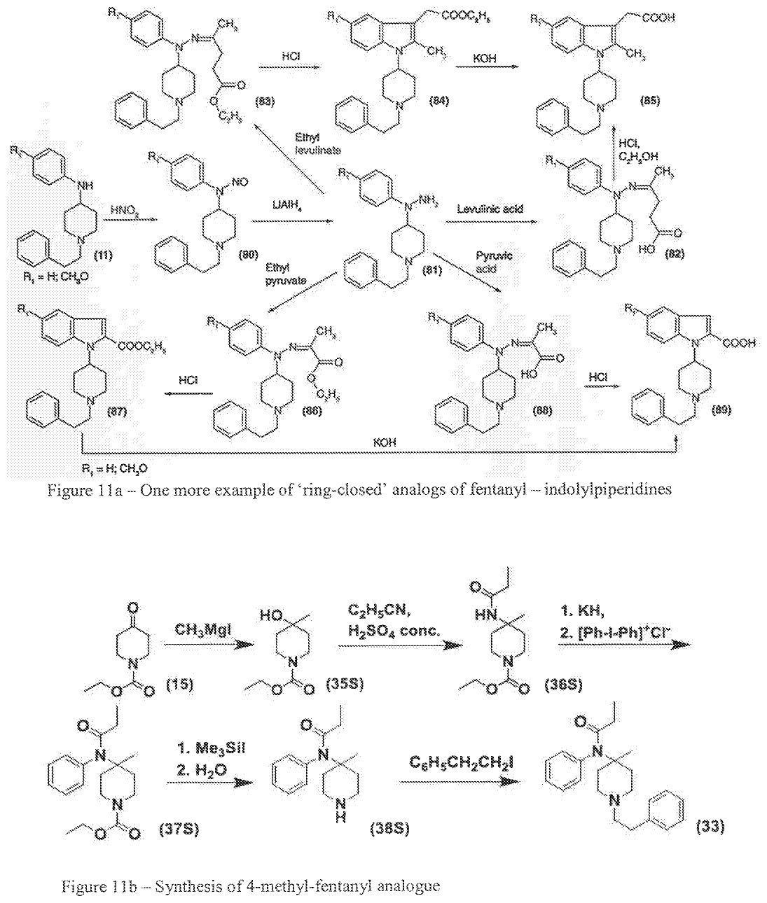

A simple and efficient synthesis of 4-methylfentanyl (32) implementing the Ritter reaction was to 4-methylpiperidin-4-ol was proposed recently (FIG. 11b) (Micovic I V, Ivanovic M D, Vuckovic S M, et at. The synthesis and preliminary pharmacological evaluation of 4-methyl fentanyl. Bioorg. Med Chem. Lett. 2000; 10(17):2011-2014). The potency of 4-methyl fentanyl (32) was found to have an ED₅₀ value of 0.0028 mg/kg in rats, which is approximately four-times greater than that of fentanyl (ED₅₀=0.0105 mg/kg), while the time peak and the duration of action are the same as those of fentanyl.

The potency of 4-methylfentanyl (32) was found to have an ED₅₀ value of 0.0028 mg/kg in rats, which is approximately four-times greater than that of fentanyl (ED₅₀ 0.0105 mg/kg), while the time peak and the duration of action are the same as those of fentanyl (FIG. 11b).

### Insertion of Substituents Other Than Methyl Into the Different Positions of the Fentanyl Structure

Any information concerning incorporation of aromatic groups into the second or third position of 4-anilidopiperidines has not been found in the literature.

The incorporation of a 4-phenyl group into 4-anilidopiperidines led to novel potent opioid analgesics with a favorable pharmacological profile. Variations in the analgesic efficacy in the series was dependent on the substituents on the piperidine nitrogen and the anilido phenyl group.

The synthesis, analgesic activity, and anesthetic properties of a series of 4-phenyl-4-anilido-piperidines and various heteroaryl substituents was carefully developed and described (FIG. 6a) (Kudzma L V, Severnak S A, Benvenga M J, et al. 4-phenyl- and 4-heteroaryl-4-anilidopiperidines. A novel class of analgesic and anesthetic agents. J. Med. Chem. 1989; 32(12):2534-2542).

Appropriately substituted anilines were reacted with 1-benzyl-4-piperidone (1), to give imines (33). The aryl group was introduced into the fourth position of piperidine ring via reaction of the obtained imine with aryllithium to afford 4-aryl-4-anilinopiperidines (34). Propionylation of obtained diamines afforded amides (35). The debenzylation of (35) was achieved by reaction with 1-chloroethyl chloroformate followed by methanolysis. The secondary amines (36) were alkylated with arylethyl halides or tosylates to give a series of compounds (37). Among them, compounds such as trefentanil (38), brifentanil (39) and 40. Within this group 40 had the highest analgesic potency (ED₅₀=0.047 mg/kg), shortest duration of action, rapid recovery of motor coordination following anesthesia doses, and greater cardiovascular and respiratory safety as compared with fentanyl and alfentanil (Gardocki J F, Yelnosky J. Some of the pharmacologic actions of fentanyl citrate and droperidol. Toxicol. Appl. Pharmacol. 1964; 6(1):48-62). A variety of other heterocyclic substitutions in both aromatic parts of fentanyl are proposed in patents (Lin B-S, Kudzma L V, Spencer H K. U.S. Pat. No. 4,791,120. 1988, Kudzma L V, Spencer H K. U.S. Pat. No. 4,801,615. 1989). Chemical modifications at the fourth position of the piperidine ring in fentanyl became an approach, which led to creation of the most powerful compounds of fentanyl series such as carfentanyl, sufentanil, alfentanil and others.

The synthesis of several derivatives of 4-arylamino-4-piperdinecarboxylic acids was reported (FIG. 7a). These acids are starting materials for the preparation of α-amino esters, ethers and ketones with strong analgesic activity (Van Daele P G, De Bruyn M F, Boey J M, et al. Synthetic analgesics: N-(1-[2-arylethyl]-4-substituted 4-piperidinyl) N-arylalkanamides. Arzneimittelforschung. 1976; 26(8):1521-1531, Janssen P A J, Van Daele G H P. U.S. Pat. No. 4,179,569. 1979). A key step in the synthesis of all mentioned compounds became one of variations of the Strecker reaction, the interaction of imines, obtained from piperidin-4-ones and aromatic amines with hydrogen cyanide and is described in FIG. 7a. The aminonitrile 41 was synthesized via interaction piperidin-4-one (7), aniline and hydrogen cyanide. Hydrolysis of the obtained nitrite to corresponding acid was performed in two steps. Treatment of 41 with sulfuric acid gave amide 42, which was hydrolyzed further to acid 43 under basic conditions—using potassium hydroxide. Alkylation of the salt of obtained acid with methyl iodide gave aminoester 44, which finally was acylated with propionic anhydride to afford the desired carfentanil 45 (Van Daele P G, De Bruyn M F, Boey J M, et al. Synthetic analgesics: N-(1-[2-arylethyl]-4-substituted 4-piperidinyl) N-arylalkanamides, Arzneimittelforschung. 1976; 26(8):1521-1531, Janssen P A J, Van Daele G H P. U.S. Pat. No. 4,179,569. 1979), a highly potent and short-acting analgesic, (lowest ED₅₀=0.00032 mg/kg) (10,000-times more potent than morphine) (Van Daele P G, De Bruyn M F, Boey J M, et al. Synthetic analgesics: N-(1-[2-arylethyl]-4-substituted 4-piperidinyl) N-arylalkanamides. Arzneimittelforschung. 1976; 26(8):1521-1531).

Carfentanil is intended only for animal use since its high potency makes it inappropriate for use in humans. Unfortunately this synthetic opioid is spreading throughout the U.S. and the world and it is more dangerous than many of the other drugs. Carfentanil, previously known by the tradename Wildnil, is a clone of fentanyl, another synthetic opioid analgesic. Both carfentanil and fentanyl are far more potent than heroin and prescription opioids like OxyContin. They are also cheaper to produce and virtually undetectable when added to street drugs such as cocaine or heroin, even fake pills. The key difference between fentanyl and carfentanil is potency. Fentanyl is 50-100 times stronger than morphine and 2 mg can be enough to cause overdose or death. Carfentanil is also 100 times stronger than fentanyl.

As mentioned, fentanyl is a second generation synthetic phenylpiperidine class opioid. It was synthesised as a solution for breakthrough pain in the 1960s and is used with other preparations in anaesthesia. Fentanyl is also used to manage chronic pain in patients with severe illnesses like cancer or those who have undergone particularly invasive surgical procedures. The illicit use of fentanyl was first uncovered in the 1970s. Although initially confined mainly to the medical community, fentanyl abuse spread quickly and prescription drug addiction is at crisis point today. Over 12 different illicitly produced fentanyl analogues, including the deadly carfentanil, have been identified in the US in recent years. Fake pills, such as counterfeit oxycodone by clandestine manufacturers, are also being laced with fentanyl and the US is being saturated with the new product. Counterfeit pills make it very difficult for users to know what they are taking; it further becomes impossible to accurately gauge the dosage and overdose is common.

Carfentanil, or carfentanyl, a structural analogue of fentanyl, continues to be a problem around the world since it's cheap and easy to make. In tests, Carfentanil is shown to be up to 4,000 times more potent than heroin. Similar in appearance to table salt, as little as 2 mg of carfentanil in one gram of heroin or cocaine is enough to potentially kill up to 50,000 people.

Because of its high potency, carfentanyl was manufactured to be a large animal tranquilizer, which could immobilize large animals very quickly. Think of a 15,000-pound wild African elephant, which is as much as 75 times the weight of a 200-pound adult man. It only takes very small quantities (as little as a 10 milligram dose) of this animal tranquilizer to sedate, or even kill, an animal of this size. This is what zoo veterinarians use and only qualified veterinarians wearing full protective gear such as mask, goggles, gloves and long sleeves can administer this drug.

Carfentanil poses significant dangers to human if taken, since it's extremely potent, easy to disguise and absorbing it on the skin or inhaling it can be fatal. Unfortunately carfentanil is now being illegally distributed on the streets, and is believed to be much more potent than all the other types of opiate substances being marketed. Dealers are now using the carfentanil opioid to cut heroin to make the effect more intense. Consider fentanyl. It's already up to 50 times more potent than heroin and up to 100 times stronger than morphine. And even at extremely low levels, fentanyl is life-threatening. In fact, it was the highest-potency opiate for human use on the market, until now. Carfentanil is 100 times more potent than fentanyl.

Now, consider the fact that it only takes a small amount of this drug to sedate a bear, elephant or other large animal. Just a couple of grains (the size of rice) of carfentanil, when injected or snorted, can cause your breathing to stop—instantly. Even the tiniest dose can come with deadly consequences. In fact, this drug is so powerful, it was referenced in the 1997 movie “Jurassic Park: The Lost World” as being used to tranquilize the Tyrannosaurus rex. The fentanyl crisis has reached international proportions with overdoses and deaths reported in the US, Canada, Europe, Australia, New Zealand and the Far East on an almost daily basis. Several different types of fentanyl, including the potentially lethal derivative carfentanil, are being illegally manufactured for sale on the streets. As well as being added to substances like cocaine and heroin, fentanyl and carfentanil can be made into pills that mimic the look of Percocet, OxyContin or other prescription drugs.

The risk of accidental overdose is significantly increased when fentanyl and carfentanil are mixed with other opioids like heroin or with cocaine, benzodiazepines or alcohol. Furthermore, illicitly-made fentanyl is more toxic than pharmaceutical opioid products and there is no way to detect how much has been added to street drugs. Unless your drugs are prescribed by a reputable pharmacy, it's almost impossible to know what they contain.

In the US alone, fentanyl-related fatalities are rising at an alarming rate; in 2016 substances containing fentanyl and fentanyl analogues caused 20,100 deaths, an increase of over 540% in just three years. The epidemic has spread across the globe and shows no sign of abating. See also Exhibits A-E in this application “Fentanyl and Carfentanyl information and help” on http://dan247.org.uk/Drug_Fentanyl.asp, “A toxic drug, more powerful than fentanyl, hits Alberta” written by Jason Markusoff Feb. 17, 2016 of Maclean's, “Is W-18 The New Dangerous Opioid On The Block” written by Megan Hesse on https://oceanbreezerecovery.org/blog/w-18/#, Fact Sheet for OSCs: Fentanyl and Fentanyl Analogs version 1.0 May 22, 2018 issued by United States Environmental Protection Agency, and Carfentanyl Critical Review Report Agenda Item 4.8 issued by Expert Committee on Drug Dependence 39^{th} meeting 2017 World Health Organization

The relative potency and selectivity of carfentanil for the 82 , κ, δ opioid receptors was determined in rat brain tissue homogenates. Carfentanil (45) was equipotent in displacing the μ and κ radioligands with IC₅₀ values of 0.0006 and 0.0008 nM, respectively, while displacing the δ ligand with IC₅₀ value of 0.75 nM. (Relative selectivity for μ/κ/δ˜1:1:1670) (Thompson R G, Menking D, Valdes J J. Opiate receptor binding properties of carfentanil. Chem. Res. Dev. Eng. Cent. Report. 1987).

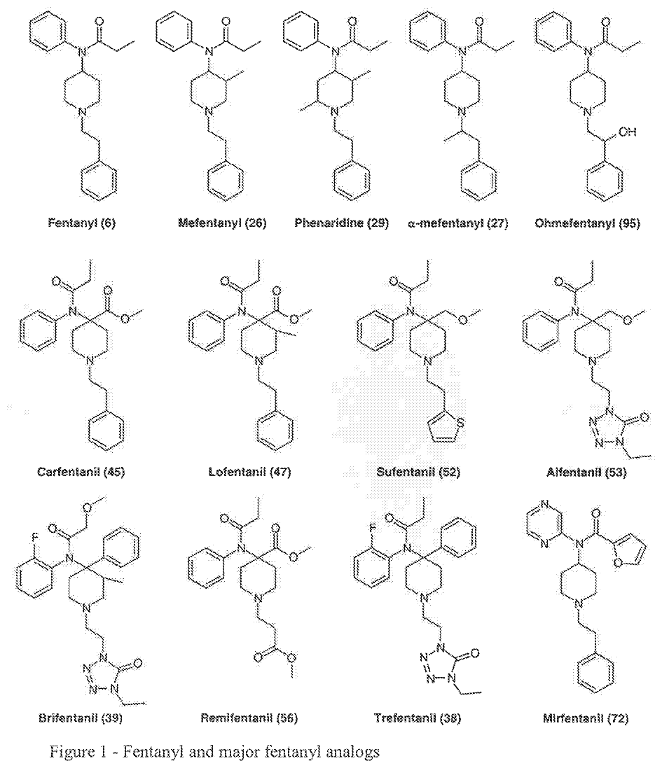

Introduction of an additional methyl group in the C-3 position of the piperidine ring resulted in another highly potent and long-acting compound lofentanil (47) (FIG. 1), which is the longest acting compound in the series (more than 8 h) with an ED₅₀ value of 0.0006 mg/kg (Van Bever W F M, Niemegeers C J E, Schellekens K H L, Janssen P A J. N-4-substituted 1-(2-arylethyl)-4-piperidinyl-N-phenylpropanamides, a novel series of extremely potent analgesics with unusually high safety margin. Arzneimittelforschung. 1976; 26(8):1548-1551).

Two very small changes in carfentanil structure, just reduction of carbonyl group in carboxy-function in the fourth position of piperidine ring transforming it to a methoxymethylene group and isosteric replacement of the phenyl ring at the phenethyl group with an heteroaromatic thienyl- and tetrazolyl rings led to two new analgesics with new properties, sufentanil (52) and alfentanil (53).

The synthesis of 4-(alkoxymethyl)piperidines was performed starting from (47) a benzyl analog of ether (44) (Niemegeers C J, Schellekens K H, Van Bever W F, Janssen P A. Sufentanil, a very potent and extremely safe intravenous morphine-like compound in mice, rats and dogs. Arzneimittelforschung. 1976; 26(8):1551-1556). Thus, 1-benzyl-4-phenylaminopiperidine-4-carboxylic acid methyl ester (47) was reduced with sodium bis-(2-methoxyethoxy) aluminium hydride (Red-Al) to give corresponding alcohol (48), sodium salt of which was selectively converted into methyl ether (49). The ether (49) was acylated with propionic anhydride to give (50). The latter was debenzylated via hydrogenation over Pd/C catalyst and the obtained amine (51) was alkylated with 2-(thiophen-2-yl)-ethyl mesylate to give desired (52) sufentanil. Sufentanil has a rapid onset of action, (ED₅₀=0.00071 mg/kg, LD₅₀=17.9 mg/kg), 4521-times more potent than morphine, and approximately five- to seven-times that of fentanyl at the time of peak effect. It has a relatively short duration of action comparable to that of fentanyl and an unusually high safety margin (LD₅₀/lowest ED₅₀=25,000).

N-alkylation of the same amine (51) with 1-(2-halogenyl)-4-ethyl-1H-tetrazol-5(4H)-one yielded another popular analgesic alfentanil (53).

The ED₅₀ of alfentanil is 0.044 mg/kg. The safety margin in rats is 1080 (LD₅₀=47.5 mg/kg i.v.). Alfentanil reaches its peak effect within 2 min after injection, and its duration of action is very short, 11 min in comparison with 30-35 min for pethidine and fentanyl, and 120 min for morphine (Janssens F, Torremans J, Janssen P A J. Synthetic 1,4-disubstituted 1,4-dihydro-5H-tetrazol-5-one derivatives of fentanyl: alfentanil (R 39209), a potent, extremely short-acting narcotic analgesic. J. Med. Chem. 1986; 29(11):2290-2297, Niemegeers C J E, Janssen P A J. Alfentanil (R 39 209)—a particularly short-acting intravenous narcotic analgesic in rats. Drug Dev. Res. 1981; 1(1):83-88).

As an analgesic in rats, alfentanil is 140-times more potent than pethidine and 72-times more potent than morphine. Alfentanil reaches its peak effect within 1 min after injection. Compared with fentanyl (6), alfentanil (53) reaches its peak effect is about four-times faster, but acts three-times shorter. While it gives less cardiovascular complications, it tends to give stronger respiratory depression. The detailed synthesis and structure-activity relationship of the series of compounds of the general formula (54) analogs of alfentanil (53) has been described (Janssens F, Torremans J, Janssen P A J. Synthetic 1,4-disubstituted 1,4-dihydro-5H-tetrazol-5-one derivatives of fentanyl: alfentanil (R 39209), a potent, extremely short-acting narcotic analgesic. J. Med. Chem. 1986; 29(11):2290-2297). N-alkylation of the amine (50) with phenethyl bromide gave R30490 (55), which has tenfold higher affinity than fentanyl for the μ receptor. R30490 is an excellent candidate to be a laboratory tool for probing determinants of μ-recognition and activation (FIG. 7a).

Analgesic activity of compounds (54) was evaluated on thermal tail withdrawal test in rats. It was found that these compounds with R═H are inactive at doses of 2.5 or 10 mg/kg. Maximal analgesic activity was found for compounds with R═COOCH₃ (carfentanil analogs) with maximal analgesic activity when R₁ represents lower alkyl groups. Compounds (53) with R═CH₂OCH₃ (sufentanil analogs) show the same activity profile as carfentanil analogs.

The ethylene group is the optimal bridge between the aromatic moiety and the nitrogen piperidine atom (R₂═R₃═H, n=0). Duration of activity, in the series of carfentanil analogs when R₁=n-C₃H₇ and R₁=c-C₃H₅ is extremely short, while at R₁═CH₃ and R₁═i-C₃H₇ compounds have an analgesic effect which last four-times longer. For sufentanil analogs (R₄═CH₂OCH₃) the duration of analgesic activity lasts between 11 and 20 min.

In an attempt to prepare novel analgesics in the fentanyl series, studies were initiated (Bagley J R, Thomas S A, Rudo F G, et al. New 1-(heterocyclylalkyl)-4-(propionanilido)-4-piperidinyl methyl ester and methylene methyl ether analgesics. J. Med. Chem. 1991; 34(2):827-841) to create new 1-(heterocyclyalkyl)-4-(propionanilido)-4-piperidinyl methyl esters and methylene methyl ethers where aromatic β-substituent (benzene, thiophene, tetrazole) at the first position of piperidine ring was replaced for a variety of other possible heterocyclic substituents.

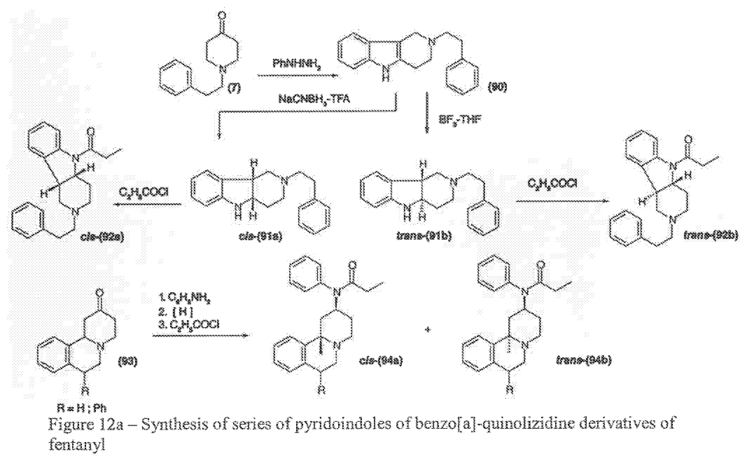

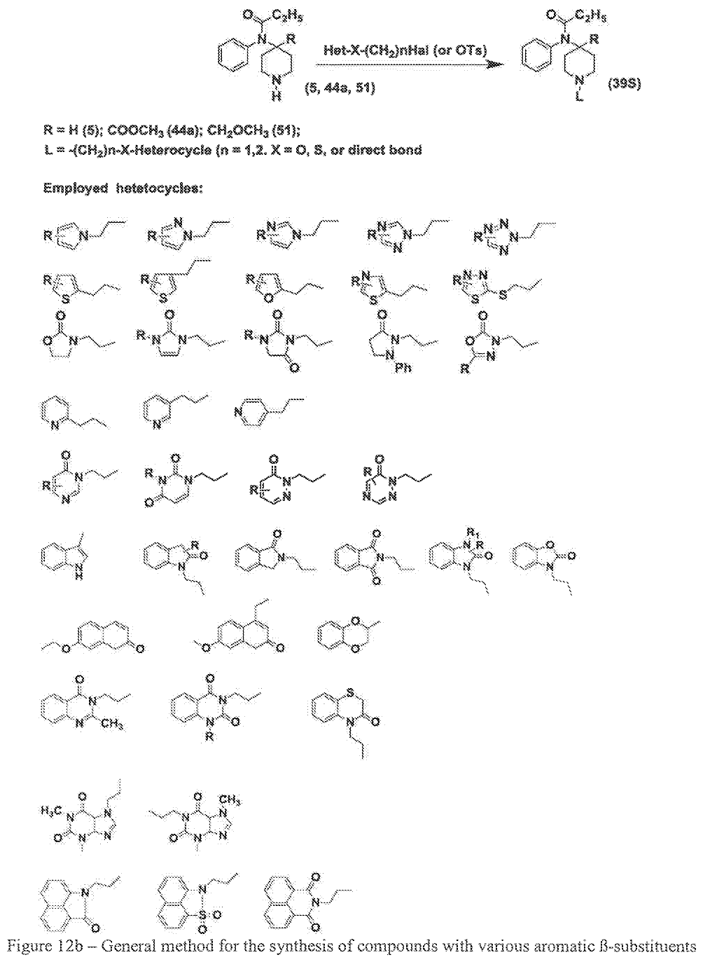

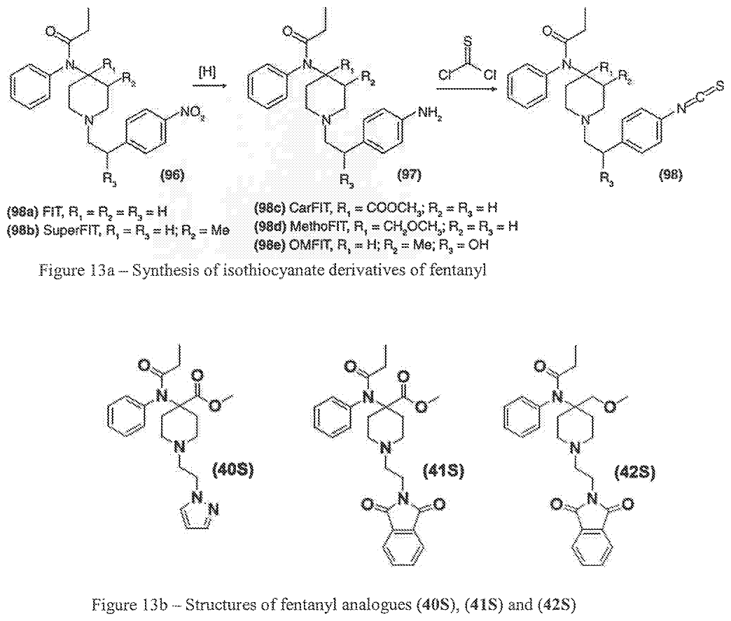

The synthesized compounds were tested in the mouse hotplate test. Most of them exhibited an analgesia (ED₅₀<1 mg/kg) superior to that of morphine. New interesting compounds like the pyrazolylethyl derivative (ED₅₀=0.0099 mg/kg), phthalimidoethyl compounds with (ED₅₀=0.056 mg/kg) and (ED₅₀=0.119 mg/kg), were obtained, which exhibited appreciable μ-opioid receptor affinity, and were more potent and short-acting analgesics and less respiratory depressants than alfentanil. In addition, some of them showed a superior motor coordination following from full anesthetic doses in the rat rotarod test (FIG. 12b and FIG. 13b).

Summarizing above, introduction of additional substituents into the fourth position of the piperidine ring of fentanyl (6) such as a carbomethoxy group gave carfentanil (45), whereas introduction of a methoxymethyl group coupled with replacement of the phenyl ring of the phenethyl with a thienyl or tetrazolyl ring led to sufentanil (52) and alfentanil (53).

The next experiments with replacement of the phenyl ring of the phenethyl group in the first position of piperidine ring was substitution for a carbomethoxy group, which brought the discovery of the ultra-short acting powerful analgesic remifentanil (56) on the market on the market (Janssen P A J, Van Daele G H P. U.S. Pat. No. 4,179,569. 1979, Kiricojevic V D, Ivanovic M D, Micovic I V, et al. An optimized synthesis of a key pharmaceutical intermediate: methyl 4-[(1-oxopropyl) phenylamino]piperidine-4-carboxylate. J. Serb. Chem. Soc. 2002; 67(12):793-802, Srimurugan S, Murugan K, Chen C. A facile method for preparation of [2H3]-sufentanil and its metabolites. Chem. Pharm. Bull. 2009; 57(12):1421-1424, Henriksen G, Platter S, Marton J, et al. Syntheses, biological evaluation, and molecular modeling of 18F-labeled 4-anilidopiperidines as μ-opioid receptor imaging agents. J. Med. Chem. 2005; 48(24):7720-7732). It is necessary to mention especially the great material on the synthesis of analogs of fentanyl summarized in (Janssen P A J, Van Daele G H P. U.S. Pat. No. 4,179,569. 1979).

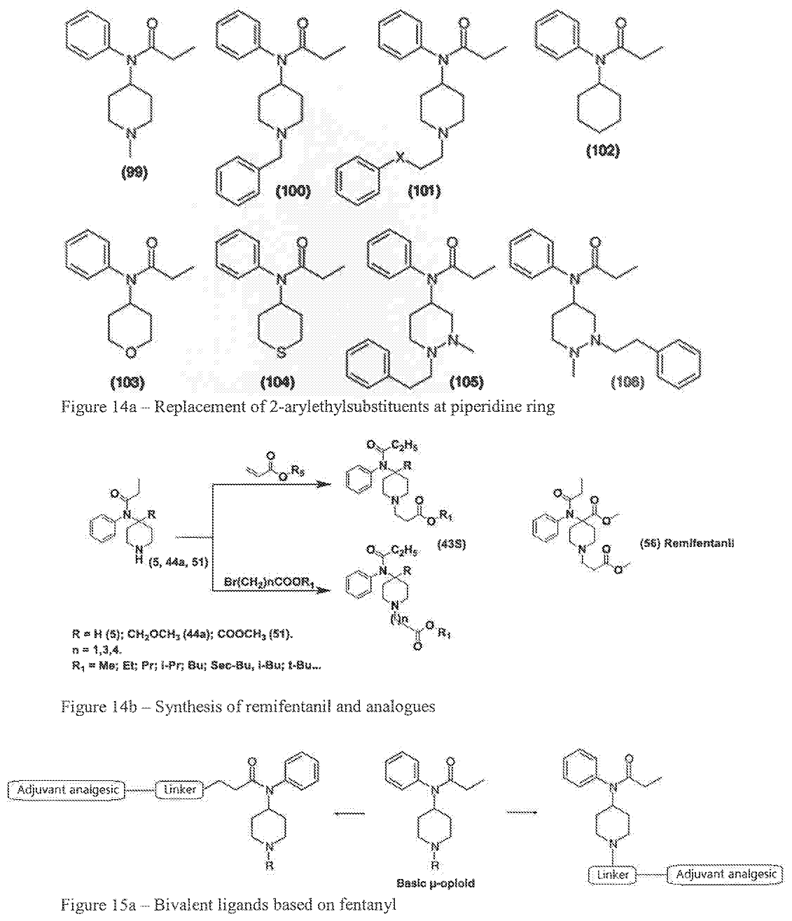

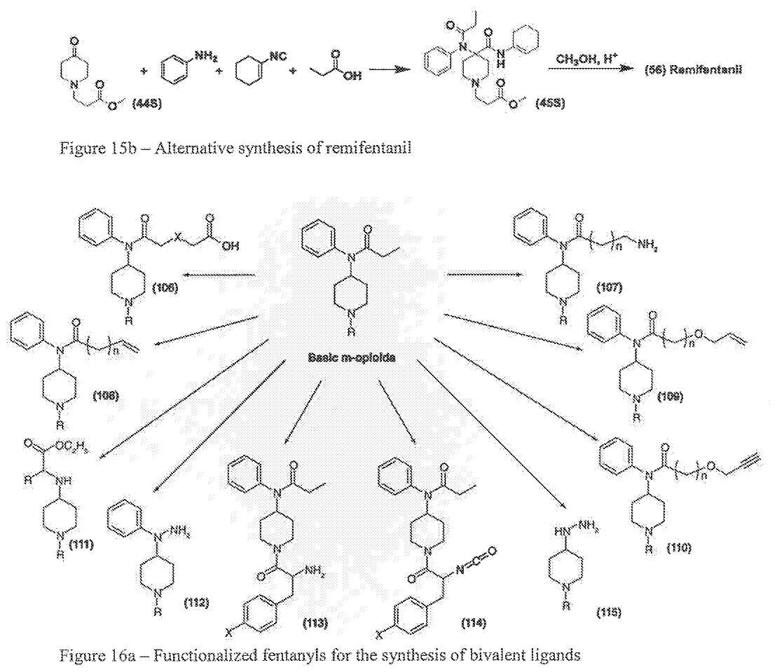

A unique drug remifentanil (56) (FIG. 1) with a high degree of analgesic potency (ED₅₀ 0.0044 mg/kg) and ultra-short duration of action (15 min.) became a clinically useful addition to the fentanyl family of analgesics (Feldman P L, James M K, Brackeen M F, et al. Design, synthesis, and pharmacological evaluation of ultrashort- to long-acting opioid analgesics. J. Med. Chem. 1991; 34(7):2202-2206, Feldman P L, James M K, Brackeen M F, Johnson M R, Leighton H. EP383.579. 1990). Later, other data appeared in literature (Cui Y, Pan L, Ning Y, Zhang K, Zheng J. Analgesic effect and time-effect relation of remifentanil. Zhongguo Yaowu Yilaixing Zazhi. 2003; 12(4):268-269. 283), according to which ED₅₀ and 95% confidence limits of analgesic effect of remifentanil on mice were 0.73 (0.64-0.84) mg/kg and 0.19 (0.12-0.31) mg/kg, respectively in hot-plate and clam-tail tests. Analgesic effects of remifentanil on rat were 2.70 (1.15-6.34) mg/kg and 5.21 (2.11-12.85) mg/kg, respectively in formaldehyde and swing-tail methods. The analgesic action of remifentanil was the strongest at 1 min after intravenous injection. The action was weakened after 6 min., and disappeared after 12 min. Remifentanil (56) occupied its own place in the arsenal of opioid analgesics and described and discussed in many pharmacological reviews (Nora F S, Fortis E, Aparecida F. Remifentanil: do we need another opioid? Rev. Bras. Anestesiol. 2001; 51(2):146-159, Battershill A J, Keating G M. Remifentanil: a review of its analgesic and sedative use in the intensive care unit. Drugs. 2006; 66(3):365-385, Scott L J, Perry C M. Remifentanil. A review of its use during the induction and maintenance of general anaesthesia. Drugs. 2005; 65(13):1793-1823, Beers R, Camporesi E. Remifentanil update: clinical science and utility. CNS Drugs, 2004; 52(6):1095-1104, Wilhelm W, Wrobel M, Kreuer S, Larsen R. Remifentanil. An update. Anaesthesist. 2003; 52(6):473-494, Rosow C E. An overview of remifentanil. Anesth. Analg. 1999; 89(4 Suppl):S1-S3, Peacock J E. Remifentanil clinical studies. Drugs Today. 1997; 33(9):619-626, Patel S S. Spencer C M. Remifentanil. Drugs. 1996; 52(3):417-427, Buerkle H D S, Van Aken H. Remifentanil: a novel, short-acting, μ-opioid. Anesth. Analg. 1996; 83(3):646-651, James M K. Remifentanil and anesthesia for the future. Exp. Opin. Invest. Drugs. 1994; 3(4):331-340), including an originally titled review “Remifentanil: do we need another opioid?” (Battershill A J, Keating G M. Remifentanil: a review of its analgesic and sedative use in the intensive care unit. Drugs. 2006; 66(3):365-385). Many modifications of the scheme of synthesis were proposed (Floegel O, Weigl U. WO2010000282. 2010, Cheng B K-M, Halvachs R E. WO2008066708. 2008, Cheng B. WO2008045192. 2008, Jacob M, Killgore J K. WO2001040184. 2001, Malaquin S, Jida M, Gesquiere J-C, et al. Ugi reaction for the synthesis of 4-aminopiperidine-4-carboxylic acid derivatives. Application to the synthesis of carfentanil and remifentanil. Tetr. Lett. 2010; 51(22):2983-2985), among which is an interesting approach that applies the Ugi reaction for the synthesis of remifentanil (56) and carfentanil (45) (FIG. 14b and FIG. 15b) (Malaquin S, Jida M, Gesquiere J-C, et al. Ugi reaction for the synthesis of 4-aminopiperidine-4-carboxylic acid derivatives. Application to the synthesis of carfentanil and remifentanil. Tetr. Lett 2010; 51(22):2983-2985).

A number of pathways for the synthesis of acyl-substituents in the fourth position of piperidine ring were envisaged (Van Daele P G, De Bruyn M F, Boey J M, et al. Synthetic analgesics: N-(1-[2-arylethyl]-4-substituted 4-piperidinyl) N-arylalkanamides. Arzneimittelforschung. 1976; 26(8):1521-1531). Acid 57 was used as starting materials for the preparation of α-amino ketones (64) via direct reaction with methyl- or butyl lithium (FIG. 8a).

However, starting amino acids with other substituents on the piperidine nitrogen atom such as benzyl, methyl or hydrogen could not he used. For this reason the acid 57 salt was reacted with ethyl chloroformate to be transferred to 1-(ethoxycarbonyl)-4-(phenylamino)piperidine-4-carboxylic acid (58), which on interaction with phosgene gave oxazolidine-2,5-dione derivative (59). Then 58 was employed in a reaction with a Grignard reagent, which led to formation of two compounds, the desired ketone 60 and piperidin-edicarboxylate (61). Hydrolysis of the mixture gave easily separable ketone (62). Alkylation on the piperidine nitrogen atom gave the keto-derivative 63, subsequent acylation of which gave the target compound 64 with very good analgesic activity (ED₅₀=0.00064-0.0013 mg/kg). For example, N-[4-acetyl-1-(2-phenylethyl)-4-piperidinyl]-N-phenylpropanamide (64 series) R₁=1-(2-phenylethyl) (ED₅₀=0.00064 mg/kg) was found to be 4900-times as potent as morphine.

The synthesis and pharmacological evaluation of another class of 4,4-disubstituted fentanyl derivatives-4-(acylamino)-4-[(acyloxy)-methyl] piperidines have been reported (Colapret J A, Diamantidis G, Spencer H K, et al. Synthesis and pharmacological evaluation of 4,4-disubstituted piperidines. J. Med. Chem. 1989; 32(5):968-974). Many efforts to selectively acylate the hydroxy-group in 48 in the presence of the anilido nitrogen failed. The synthesis of target compounds was accomplished via protecting the hydroxyl group in 48 via formation of trimethylsilyl ether 65. After the acylation of anilido nitrogen in 65, attempts to unmask the hydroxy-group in 66 unexpectedly afforded product of intramolecular esterification 68. (During the hydrolysis of the O-trimethylsilyl ester acyl group migration took place). After a second acylation of the anilido nitrogen (69) followed by debenzylation (70) and further alkylation with arylethyl halogenides the desired products (71) were obtained (Colapret J A, Diamantidis G, Spencer H K, et al. Synthesis and pharmacological evaluation of 4,4-disubstituted piperidines. J. Med. Chem. 1989; 32(5):968-974). The choice of the N-arylethyl substituent (phenyl, thienyl, tetrazol-on-il, phtalimidoil) was dictated by literature data (FIG. 9a).

Another set of analog compounds (Colapret J A, Diamantidis G, Spencer H K, et al. Synthesis and pharmacological evaluation of 4,4-disubstituted piperidines. J. Med. Chem. 1989; 32(5):968-974) was prepared by the condensation of aminoalkohol (48) with 1,1′-carbonyldiimidazole and the obtained adduct was transformed to series of carbonates (FIG. 16b).

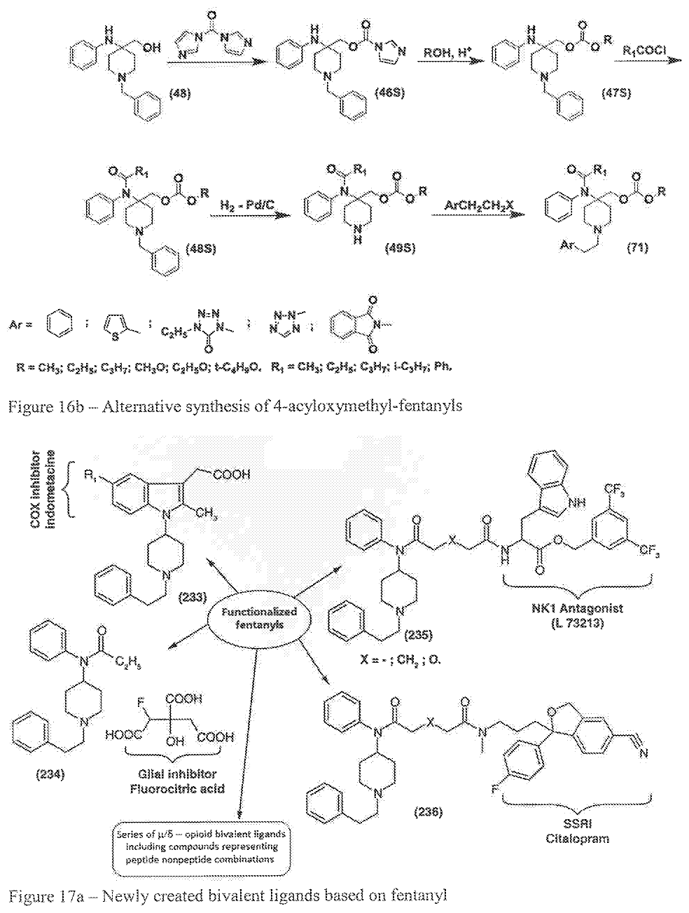

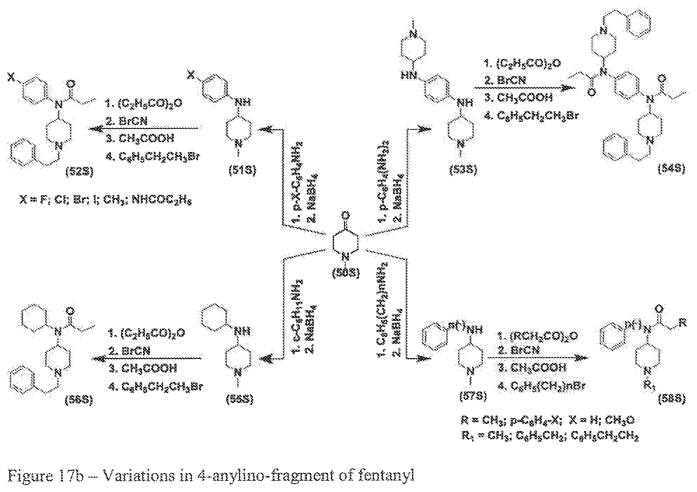

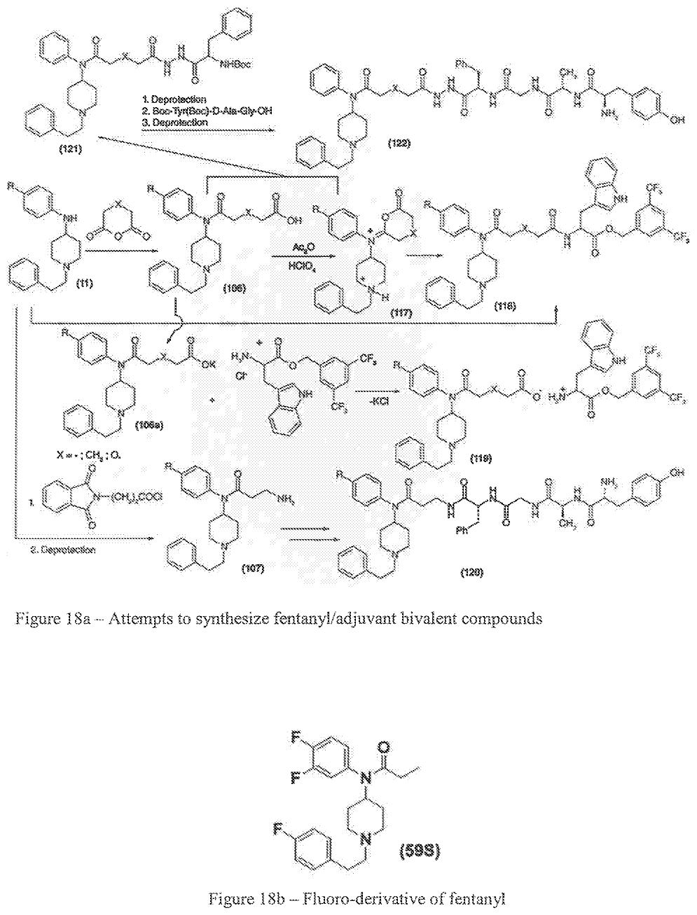

Groups of fentanyl analogs with modified 4-anilido-fragment have been synthesized. p-F, I, and CH₃ derivatives, the cyclohexyl analog, analogs with phenyl group of 4-anilido-fragment distanced from the fourth position of the piperidine ring have been described. Among compounds of that series all N-benzyl derivatives were inactive. All N-phenethyl analogs retained reasonable levels of activity less than that of fentanyl but more than that of morphine (Casy A F, Huckstep M R. Structure-activity studies of fentanyl. J, Pharm. Pharmacol. 1988; 40(9):605-608, Peters D, Eriksen B L, Munro G N, Oestergaard E. WO2009077584. 2009). The traditional ortho-substituents (Cl, F, Me, MeO) (Janssen P A J. U.S. Pat. No. 3,164,600. 1965, Kudzma L V, Severnak S A, Benvenga M J, et al. 4-phenyl- and 4-heteroaryl-4-anilidopiperidines. A novel class of analgesic and anesthetic agents. J. Med. Chem. 1989; 32(12):2534-2542, Casy A F, Huckstep M R. Structure-activity studies of fentanyl. J. Pharm. Pharmacol. 1988; 40(9):605-608, Peters D, Eriksen B L, Munro G N, Oestergaard E. WO2009077584. 2009) in the anilino-fragment of fentanyl do not act dramatically on the activity of the parent compound fentanyl. Significant changes were observed in two cases. Substitution with an —OH group brings more than a tenfold loss of activity. Introduction of a NO₂-group causes more than a 1000-fold loss of activity, equal to removal of the N-propionyl group in fentanyl (FIG. 17b and FIG. 18b). Another research approach was based on modifications of the 4-anilido-fragment and its replacement by a variety of heterocycles (Bagley J R, Wynn R L, Rudo F G, et al. New 4-(heteroanilido)piperidines, structurally related to the pure opioid agonist fentanyl, with agonist and/or antagonist properties. J. Med. Chem. 1989; 32:663-671).

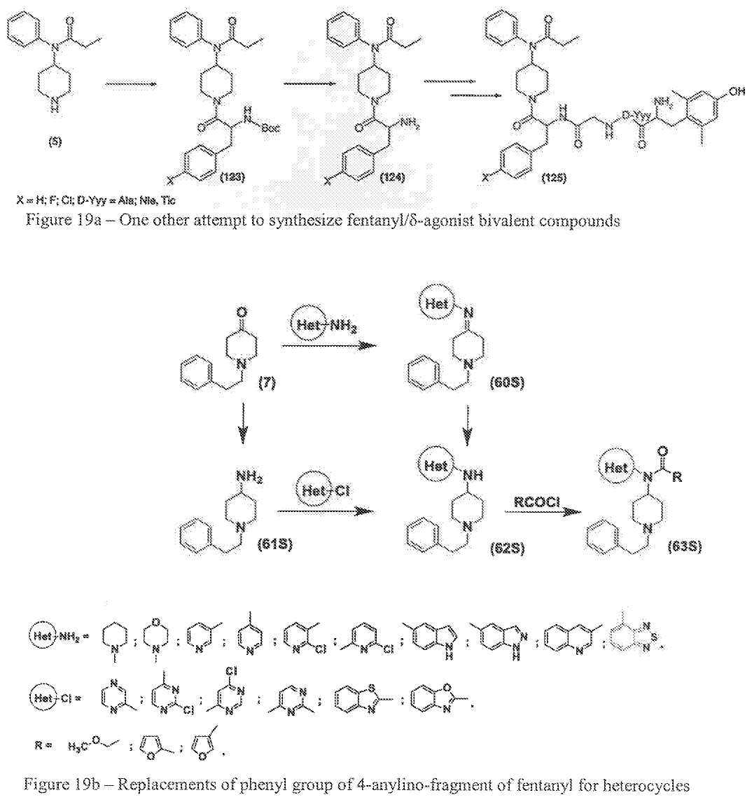

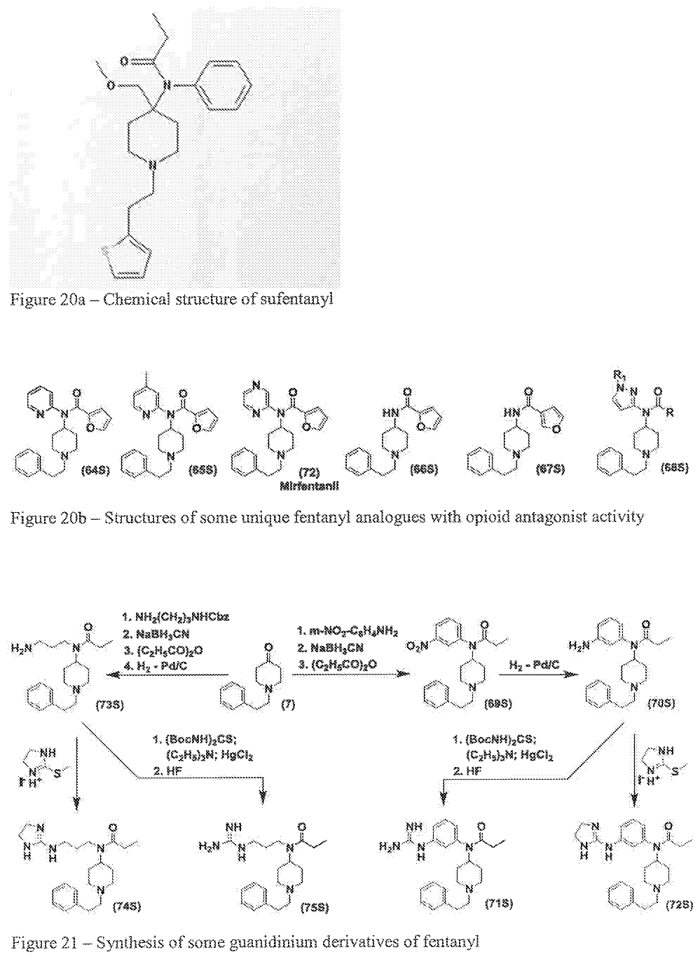

Observations that have been done in this research allowed one to make important conclusions. One of them is substitution of the phenyl ring of the propionanilido group of fentanyl for heterocycles, resulted in a significant diminution of analgesic activity. The sole exception was the 2-pyridino derivative, which shows agonistic activity comparable with fentanyl. Another observation deserves much more attention. The compounds of the described series were also screened as opioid antagonists. The majority of these compounds (80%) were morphine antagonists, and they selectively antagonized respiratory depression. The compounds with the 2-pyridinyl, 4-pyridinyl and 2-pyrimidinyl rings were found to be inactive as antagonists. Variations of an acyl-chain within the amido-substructure of 4-(heteroanilido)-piperidines likely play a larger role. Among the compounds, bearing a methoxymethyl chain attached to the amide carbonyl group opioid antagonists were not found. In contrast, 2- or 3-furyl compounds were antagonists. Moreover, most furan-containing compounds selectively reverse respiratory depression. Two compounds that were antagonists, displayed different antagonistic profiles. 4-ethyl-2-pyridinyl compound resembled naloxone in inhibiting both morphine-induced analgesia and respiratory depression, while 2-pyrazine derivative mirfentanil (72) (FIG. 1) (ED₅₀=0.07 mg/kg, rat tail-flick test), inhibited morphine analgesia slightly, but completely reversed respiratory depression. These findings are difficult to explain. Another important observation was that antagonistic activity remains even in the absence of a 4-(heteroanilido) substituent, but when the furan group was removed, there was no activity (FIG. 19b and FIG. 20b) (Bagley J R, Wynn R L, Rudo F G, et al. New 4-(heteroanilido)piperidines, structurally related to the pure opioid agonist fentanyl, with agonist and/or antagonist properties. J. Med. Chem. 1989; 32:663-671).

Making parallel comparisons of chemical structures of opioid agonists, it is possible to conclude that practically every chemical class of compounds with opioid-agonist activity has a structurally similar opioid-antagonist compound. Agonist-antagonist transformation in these cases takes place as a result of small changes in the structure of the agonist. The only exceptions, where the corresponding change for agonist-antagonist transformations has not been found are the compounds of the fentanyl series.

These structurally unique fentanyl analogs provide a new gate into the area of creation of new powerful opioid antagonists (Bagley J R, Wynn R L, Rudo F G, et al. New 4-(heteroanilido)piperidines, structurally related to the pure opioid agonist fentanyl, with agonist and/or antagonist properties. J. Med. Chem. 1989; 32:663-671, Wynn R L, Bagley J R, Spencer H K, Spaulding T C. Evaluation of the morphine reversal actions and antinociceptive activity of a new class of opiate antagonists structurally related to fentanyl. Drug Develop. Res. 1991; 22(2):189-495). The possibility of creation of opioid antagonists in the fentanyl has been patented (Vartanian R S, Vartanian S A, Martirosyan V O, et al. SU1139125. 1984).

Other fentanyl analogs in which the benzene ring of the propioanilido group was changed to a heterocyclic substituent, particularly for phenylpyrazole group also have been described (Jagerovic N, Cano C, Elguero J. et al. Long acting fentanyl analogs: synthesis and pharmacology of N-(1-phenylpyrazolyl)-N-(1-phenylalkyl-4-piperidyl)propanamides. Bioorg. Med. Chem. 2002; 10(3):817-827, Giron R, Abalo R, Goicoechea C, et al. Synthesis and opioid activity of new fentanyl analogs. Life Sci. 2002; 71(9):1023-3104, Jimeno M L, Alkorta I, Cano C, et al. Fentanyl and its analog N-(1-phenylpyrazol-3-yl)-N-[1-(2-phenylethyl)-4-piperidyl]propanamide: 1H- and 13C-NMR spectroscopy, x-ray crystallography, and theoretical calculations. Chem. Pharm. Bull. 2003; 51(8):929-934, Goicoechea C, Sanchez F, Cano C, et al. Analgesic activity and pharmacological characterization of N-[1-phenylpyrazol-3-yl]-N-[1-(2-phenethyl)-4-piperidyl] propenamide, a new opioid agonist acting peripherally. Eur. J. Pharmacol. 2008; 595(1-3):22-29). Obtained compounds showed more potent analgesic properties than morphine, but less than fentanyl with longer duration of action.

It was proposed that imidazoline receptor agonists combined with opioid agonists could produce antinociceptive synergy. Thus fentanyl derivatives that incorporate guanidinium and 2-aminoimidazolinium groups were designed and synthesized, which incorporate both μ-opioid and I₂-imidazoline receptor pharmacophores (Montero A, Goya P, Jagerovic N, et al. Guanidinium and aminoimidazolinium derivatives of N-(4-piperidyl)propanamides as potential ligands for μ opioid and I2-imidazoline receptors: synthesis and pharmacological screening. Bioorg. Med. Chem. 2002; 10(4):1009-1018, Dardonville C, Jagerovic N, Callado L E, Meana J J. Fentanyl derivatives bearing, aliphatic alkaneguanidinium moieties: a new series of hybrid molecules with significant binding affinity for mu-opioid receptors and I2-imidazoline binding sites. Bioorg. Med. Chem. Lett. 2004; 14(2):491-493). This publication was likely the first attempt for creation of bivalent ligands based on fentanyl as the μ-component. Binding assays indicate that guanidinium compounds are still potent μ-opioid ligands similar to fentanyl, but display moderate analgesic properties in vivo. The results for the I₂-imidazoline receptor are less significant and the obtained compounds showed only low affinity (FIG. 21).

Attempts to synthesize 3-methoxy- and 3-carbmethoxy-analogs of fentanyl should be noted. 3-methoxy-fentanyl analog has been prepared by traditional scheme (ketone-imine-amine-amide sequence) starting from 1-(2-phenylethyl)-piperidine-4-one (7), which was oxidized to give 3-hydroxy-piperidine-4-one dimethyl ketal, which was methylated to give 3-methoxy-piperidine-4-one ketal, converted to 3-methoxy-piperidine-4-one, which was further subjected to transformation to 3-methoxy-fentanyl. The effective dose (ED₅₀=0.00064 mg/kg) was obtained for the cis-isomer of synthesized compound (Lalinde N L, Moliterni J, Spencer H K. U.S. Pat. No. 4,994,471. 1991).

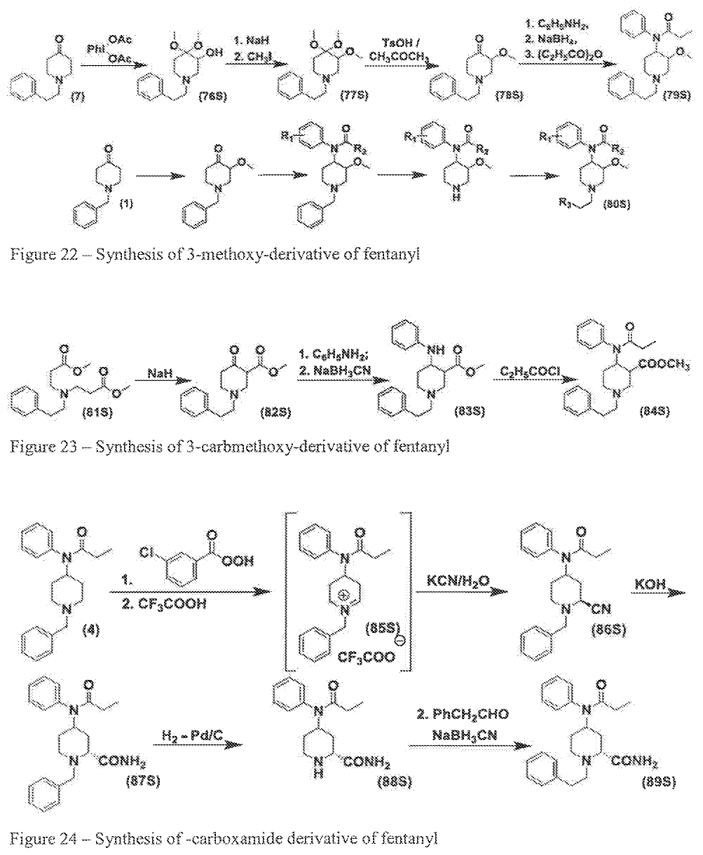

Using the regular scheme for the synthesis, starting from 3-methoxy-N-benzylpiperidine-4-one and separating on different stages cis- and trans-isomers, led to the synthesis of a plethora of highly active analgesics (ED₅₀=0.00046-0.0019 mg/kg) (FIG. 22).

The synthesis of 3-carbomethoxy fentanyl or iso-carfentanil has been accomplished starting from 3-carbomethoxy-1-(2-phenylethyl)-piperidine-4-one. Both (±) cis-(carbmethoxy group orientated axial−ED₅₀=0.023 mg/kg) and (±) trans-(carbmethoxy group orientated equatorial−ED₅₀=0.1 mg/kg) isomers of separated 3-carbomethoxy-fentanyl revealed significant but substantially reduced potency compared with fentanyl (ED₅₀=0.011 mg/kg) (FIG. 23) (Micovic I V, Ivanovic M D, Vuckovic S, et al. 3-Carbomethoxy fentanyl: synthesis, pharmacology and conformational analysis. Het. Comm. 1998; 4(2):171-179, Vuckovic S, Prostran M, Ivanovic M, et al. Antinociceptive activity of novel fentanyl analog iso-carfentanyl in rats. Jpn. J. Pharmacol. 2000; 84:188-495).

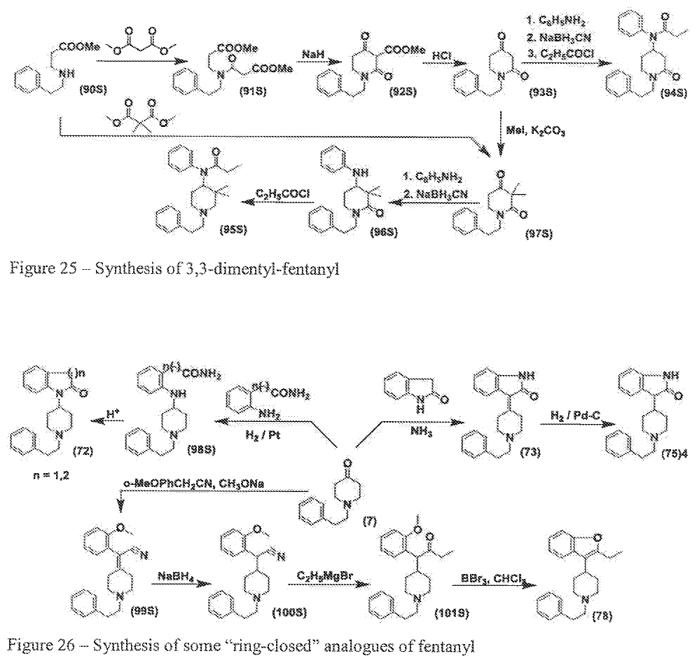

Attempts to functionalize the second position of fentanyl have been made. For example, (2R)-1-phenethyl-4-(N-phenylpropionamido) piperidine-2-carboxamide has been synthesized, which was 110- and 450-times less potent than fentanyl in the GPI and MVD assays, respectively (FIG. 24 and FIG. 25) (Essawi M Y H, Portoghese P S. Synthesis and evaluation of 1- and 2-substituted fentanyl analogs for opioid activity. J. Med. Chem. 1983; 26(3):348-352). Other types of functionalization of the second position of the piperidine ring have been examined and lactam analogs of fentanyl were synthesized (Micovic I V, Roglic G M, Ivanovic M D, et al. The synthesis of lactam analogs of fentanyl. J. Chem. Soc. Perkin Trans. 1996; 1(16):2041-2050, Micovic I V, Roglic G M, Ivanovic M D, et al. The synthesis of 3,3-dimethylfentanyl and its lactam analog. J. Serb. Chem. Soc. 1996; 61(10):849-857), but pharmacological studies were not given.

### Conformationally Restricted Analogs of Fentanyl

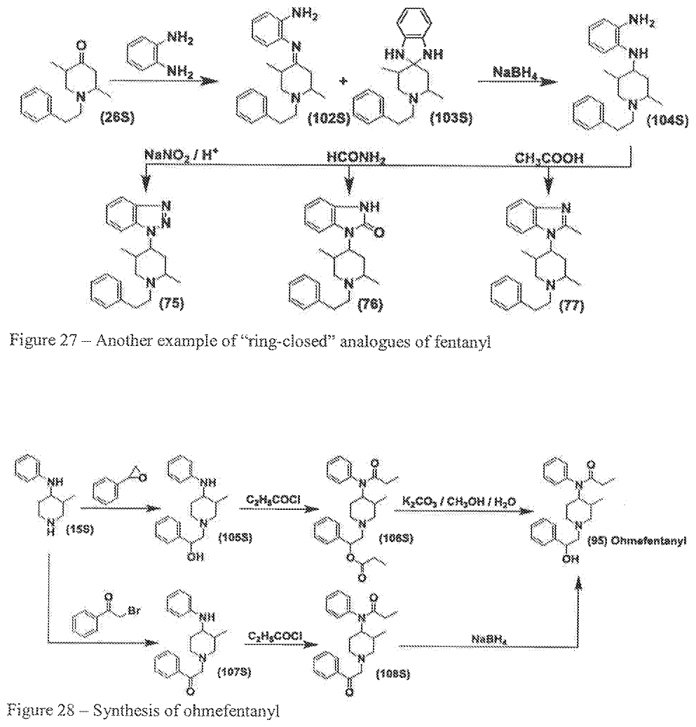

A number of ‘ring-closed’ analogs of fentanyl (72-79) were prepared (FIG. 10a). The first publication is probably that which described (4-piperidinyl)-2-indolinones n=1) and quinolinones (n=2) (72). The synthetic approaches are very simple (FIG. 26 and FIG. 27) (Klein W, Back W, Mutschler E. Potential analgesics. 3. 1-(4-piperidinyl)-2-indolinones and -3,4-dihydrocarbostyrils. Arch. Pharm. 1974; 307(5):360-366, Walker G N, Smith R T, Weaver B N. Synthesis of new 3-(pyridylmethylene)-3-(pyridylmethyl)-3-(piperidylmethyl)-, and 3-(β-alkylaminoethyl)-2-indolinones. The reduction of isoindogenides, nitro compounds, and pyridines in a series of 2-indolinones. J. Med. Chem. 1965; 8(5):626-637, Lobbezoo M W, Soudijn W, Van Wijngaarden I. Opiate receptor interaction of compounds derived from or structurally related to fentanyl. J. Med. Chem. 1981; 24(7):777-782, Vartanyan R S, Martirosyan V O, Vlasenko E V, Ovasapyan A A. Synthesis and biological activity of 1-substituted benzimidazole and benztriazole derivatives. Khim. Farm. Zh. 1982; 16(8):947-951, Kudzma L V, Evans S M, Turnbull S P, et al. Octahydro-1,2,3,4,4a,5,11,11a-pyrido3,4-c1,5benzoxazepines conformationally restricted fentanyl analogs. Bioorg. Med. Chem. Lett. 1995; 5(11):1177-1182, Van Dyke J W, Jr, Havera H J, Johnson R D, et al. Cardiovascular activity of some substituted 2-aminobenzoquinolizines. J. Med. Chem. 1972; 15(1):91-94, Maryanoff B E, McComsey D F, Taylor R J, et al. Synthesis and stereochemistry of 7-phenyl-2-propionanilidobenzo[a] quinolizidine derivatives. Structural probes of fentanyl analgesics. J. Med. Chem. 1981; 24(1):79-88, Vardanyan R, Vijay G, Nichol G S, et al. Synthesis and investigations of doublepharmacophore ligands for treatment of chronic and neuropathic pain. Bioorg. Med. Chem. 2009; 17(14):5044-5053). The affinities are much less than that of fentanyl. From 500- to 800-times, until a complete loss of analgesic activity. In attempts to synthesize bivalent analgesics another series of ‘ring-closed’ analogs of fentanyl that combined fentanyl and indometacine structures some (4-piperidinyl)-indoles have been synthesized (Kudzma L V, Evans S M, Turnbull S P, et al. Octahydro-1,2,3,4,4a,5,11,11a-pyrido3,4-c1,5benzoxazepines conformationally restricted fentanyl analogs. Bioorg. Med. Chem. Lett. 1995; 5(11):1177-1182).

For creation of this series N-(piperidin-4-yl)-N-phenyl-hydrazines (81) were synthesized (FIG. 11a) starting from 4-anilinopiperidines (11), which were nitrosylated to the N-nitroso compounds (80) and then hydrogenated to the desired 4-piperidylphenylhydrazines (81). Then they were reacted with levulinic acid or its esters and the hydrazones 82 and 83 underwent Fishertype reactions, which led to indole derivatives, a series of indometacine analogs 84 and 85.

Docking experiments of obtained molecules to their corresponding receptors were performed that predicted high COX-2 inhibitor activity compounds equal to indometacine itself and opioid activity two- to three-times higher than that of the fentanyl. However biological assays data were not consistent with the results from the molecular modeling. The synthesized compounds did not show any significant analgesic activity in the entire series (Vardanyan R, Vijay G, Nichol G S, et al. Synthesis and investigations of double pharmacophore ligands for treatment of chronic and neuropathic pain. Bioorg. Med. Chem. 2009; 17(14):5044-5053). Another series of indolylpiperidines 87 and 89 have been synthesized from phenylhydrazones 86 and 88 obtained from the same 4-piperidylphenylhydrazines (81). These compounds also did not show any significant analgesic activity for the entire series (FIG. 11a) [Vardanyan R S, Unpublished Data].

It is well known that for the most active representatives of the fentanyl series the aniline phenyl is perpendicular to the plane of the amide function (Tollenaere J P, Moereels H, Raymaekers L A. Atlas of the Three-Dimensional Structure of Drugs, Vol. 1. Amsterdam, The Netherlands: Elsevier/North-Holland, Inc; 1979, Koch M H J, de Ranter C J, Rolies M, Dideberg O. N-[4-(methoxymethyl)-1-(2-phenylethyl)-4-piperidinyl]-N-phenylpropanamide. Acta Crystallogr. Sect. B. 1976; 32:2529-2531). For the whole series of ‘ring-closed’ compounds the perpendicular conformation of the anilido-moiety is excluded (Lobbezoo M W, Soudijn W, Van Wijngaarden I. Opiate receptor interaction of compounds derived from or structurally related to fentanyl. J. Med. Chem. 1981; 24(7):777-782). It can be suggested that these prepared compounds will bind poorly to opiate receptors, and that is the main reason for the absence of analgesic activity.

There is another series of ‘semi-rigid’ compounds where conformational mobility of pharmacophore groups is restricted and the first representatives are the pyridoindoles. These compounds have been synthesized from tetrahydro-γ-carboline (90), which was synthesized by Fischer condensation of piperidine-4-one (7) with phenylhydrazine (Spickett W. Compounds affecting the central nervous system. II. Substituted 1,2,3,4-tetrahydropyrido [4,3-b] indoles. J. Med. Chem. 1966; 9(3):436-438, Berger J G, Davidson F, Langford G E. Synthesis of some conformationally restricted analogs of fentanyl. J. Med. Chem. 1977; 20(4):600-602). Compounds 92a and 92b were inactive in the mouse phenylquinone writhing test at doses of up to 130 mg/kg (FIG. 12a).

Another series of conformationally restricted analogs of fentanyl, of propionanilidobenzo[a]-quinolizidine derivatives 94a and 94b (FIG. 12a), were synthesized starting from hexahydro-pyridoisoquinolin-2-ones (93) and tested for analgesic activity and affinity for the opiate receptor of rat brain. These compounds displayed very weak analgesic activity (FIG. 12a) (Spickett W. Compounds affecting the central nervous system. II. Substituted 1,2,3,4-tetrahydropyrido[4,3-b] indoles. J. Med. Chem. 1966; 9(3):436-438, Berger J G, Davidson F, Langford G E. Synthesis of some conformationally restricted analogs of fentanyl. J. Med. Chem. 1977; 20(4):600-602). In addition, for the above discussed ‘semi-rigid’ derivatives it might be concluded that the analgesic activity in the fentanyl series does not ‘tolerate’ rigidity of whole molecule and is highly dependent on stereochemical factors.

### Substituents in the Side Chain in the α- & β-Positions of the Basic Nitrogen

Insertion of a methyl group in the N-substituent in the α-position to the basic nitrogen, also displayed high activity close to that of fentanyl, as mentioned above (Van Bever W F M, Niemegeers C J E, Janssen P A J. Synthetic analgesics. Synthesis and pharmacology of the diastereoisomers of N-[3-methyl-1-(2-phenylethyl)-4-piperidyl]-N-phenylpropanamide and N-[3-methyl-1-(1-methyl-2-phenylethyl)-4-piperidyl]-N-phenylpropanamide. J. Med. Chem. 1974; 17(10):1047-1051). According to the first fentanyl patent (Janssen P A J. U.S. Pat. No. 3,164,600. 1965) compound with methyl group in the β-position to the basic nitrogen has no advantages on fentanyl itself. Insertion of hydroxyl group in the β-position to the basic nitrogen in general enhances the potency of compounds of these series. But the most impressive results were obtained with the synthesis of ohmefentanyl (95), one of the most potent opioids known (FIG. 1 and FIG. 26). Ohmefentanyl had an ED₅₀ value of 0.0002 mg/kg. For the four isolated stereoisomers of ohmefentanyl-(±)-cis-N-[1-(2-hydroxy-2-phenylethyl)-3-methyl-4-piperidyl]-N phenylpropanamides: ED₅₀ values are: 0.0001 mg/kg for the 95a (2S,3R,4S) isomer; 0.0013 mg/kg for the 95b (2R,3R,4S) isomer; 0.08 mg/kg for the 95c (2R,3S,4R) isomer and 2.1 mg/kg for the 95d (2S,3S,4R) isomer.

The binding studies showed that the highest affinity and selectivity for the μ receptors were isomers 95b and 95c. At the same time, obtained data on the mouse vas deferens test showed potencies in the order 95a>95b>95c>95d with concentrations in the fentomolar range. According to the mouse data, isomer 95a was 21,000-times more potent than 95d. Isomers 95b and 95c had similar opiate activities in vivo. The potency of 95a was from 20,000- to 50,000-times higher than that of morphine, which makes this isomer one of the most potent opiates known (FIG. 28 and FIG. 29) (Wang Z-X, Zhu Y-C, Jin W-Q, et al. Stereoisomers of N-[1-(2-hydroxy-2-phenylethyl)-3-methyl-4-piperidyl]-N-phenylpropanamide: synthesis, stereochemistry, analgesic activity, and opioid receptor binding characteristic. J. Med. Chem. 1995; 38(18):3652-3659, Brine G A, Stark P A, Liu J Y, et al. Enantiomers of diastereomeric cis-N-[1-(2-hydroxy-2-phenylethyl)-3-methyl-4-piperidyl]-N-phenylpropanamides: synthesis, x-ray analysis, and biological activities. J. Med. Chem. 1995; 38(9):1547-1557).

### Substituents in the Aryl Group in the 1-(2-Arylethyl) Moiety of Fentanyl

For variations of the character and substituents of the aryl group in the 1-(2-arylethyl) moiety in the fentanyl family, interesting changes took place when an isothiocyanate group was inserted into the phenyl group with the goal to synthesize irreversible ligands for opioid receptors (Rice K C, Jacobson A E, Burke T R, Jr, et al. Irreversible ligands with high selectivity toward δ or μ opiate receptors. Science. 1983; 220(4594):314-316). The synthesis of the alkylating fentanyl derivatives was performed (FIG. 13a) (Burke T R, Jr, Bajwa B S, Jacobson A E, et al. Probes for narcotic receptor mediated phenomena. 7. Synthesis and pharmacological properties of irreversible ligands specific for μ or δ opiate receptors. J. Chem. 1984; 27(12):1570-1574, Burke T R, Jr, Jacobson A E, Rice K C, et al. Probes for narcotic receptor mediated phenomena. 12 cis-(+)-3-methyl-fentanyl isothiocyanate, a potent site-directed acylating agent for δ opioid receptors. Synthesis, absolute configuration, and receptor enantioselectivity. J. Med. Chem. 1986; 29(6):1087-1093).

The analgesic activity and opioid receptor binding affinities were examined for the isothiocyanate fentanyl (98a), 3-methylfentanyl (98b), carfentanil (98c), 4-methoxymethylfentanyl (98d), and ohmefentanyl (98e). In vivo antinociceptive activity was examined using the mouse hot plate test, and selectivity for opioid receptors was detected using binding assays. Fentanyl (ED₅₀ not found), carfentanil (ED₅₀=0.00041 mg/kg), and 4-methoxymethylfentanyl (ED₅₀=0.00078 mg/kg), showed ED₅₀ lower than those of their parent compounds. 3-methylfentanyl (ED₅₀=0.33 mg/kg), and ohmefentanyl (ED₅₀=0.0018 mg/kg) were stronger than the parent compounds. However the selectivity of all these compounds for δ receptors increased (Burke T R, Jr Bajwa B S, Jacobson A E, et al. Probes for narcotic receptor mediated phenomena. 7. Synthesis and pharmacological properties of irreversible ligands specific for μ or δ opiate receptors. J. Med. Chem. 1984; 27(12):1570-1574, Burke T R, jr, Jacobson A E, Rice K C, et al. Probes for narcotic receptor mediated phenomena. 12 cis-(+)-3-methyl-fentanyl isothiocyanate, a potent site-directed acylating agent for δ opioid receptors. Synthesis, absolute configuration, and receptor enantioselectivity. J. Med. Chem. 1986; 29(6):1087-1093, Chen B-Y, Jin W-Q, Chen Jie, et al. Analgesic activity and selectivity of isothiocyanate derivatives of fentanyl analogs for opioid receptor. Life Sci. 1999; 65(15):1589-1595).

### Replacement of Anilino-Moiety for Benzylamino Group

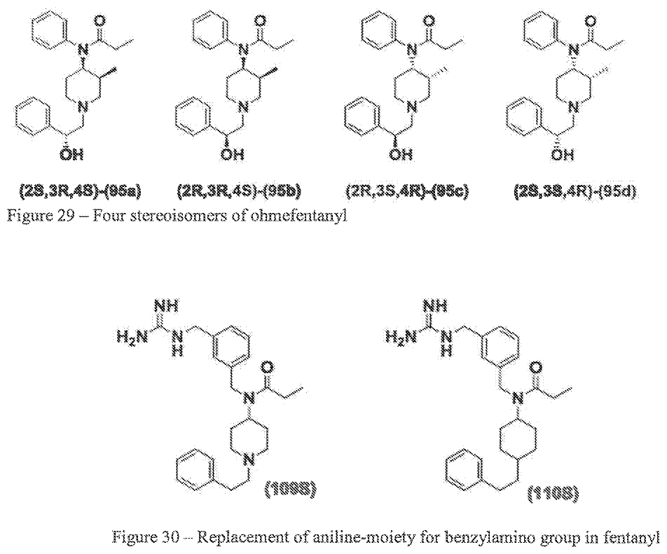

In continuation of studies devoted to involvement of imidazoline binding site in the modulation of analgesia, studies on creation of hybrid (bivalent) molecules bearing potent opioid and I2-imidazoline binding site ligands (Montero A, Goya P, Jagerovic N, et al. Guanidinium and aminoimidazolinium derivatives of N-(4-piperidyl)propanamides as potential ligands for μ opioid and I2-imidazoline receptors: synthesis and pharmacological screening. Bioorg. Med. Chem. 2002; 10(4):1009-1018, Dardonville C, Jagerovic N, Callado L F, Meana J J. Fentanyl derivatives bearing aliphatic alkaneguanidinium moieties: a new series of hybrid molecules with significant binding affinity for mu-opioid receptors and I2-imidazoline binding sites. Bioorg. Med. Chem. Lett. 2004; 14(2):491-493) has been continued and another sensitive change in fentanyl structure has been done. The phenyl moiety in anilino-fragment has been moved to one methylene group to give attractive compounds with a propionbenzylamino group (Dardonvilie C, Fernandez-Fernandez G, Gibbons S-L, et al. Synthesis and pharmacological studies of new hybrid derivatives of fentanyl active at the μ-opioid receptor and I2-imidazoline binding sites. Bioorg. Med. Chem. 2006; 14(19):6570-6580). The obtained compound showed no affinity to I₂-imidazoline binding site (Kᵢ>10,000 nM), but displayed surprisingly high μ-opioid binding (Kᵢ=0.0098 nM) (on membranes). This observation brought to another study, synthesis and investigation of its ‘carba’-analogs. Compound devoid nitrogen atom in piperidine ring of fentanyl was synthesized and showed 25-fold reduced receptor binding affinities as compared with piperidine nitrogen containing but, surprisingly it retained its opioid agonist activity (Weltrowska G, Chung N N, Lemieux C, et al. “Carba”-analogs of fentanyl are opioid receptor agonists. J. Med. Chem. 2010; 53(7):2875-2881). Moreover, in the same paper it was shown that the propionbenzylamino fentanyl analog has not only high μ-opioid binding (Kᵢ=0.448 nM), but also had high κ receptor binding affinity (Kᵢ=0.536 nM) (FIG. 30).

### Replacement of 2-Arylethyl Substituents at Piperidine Ring

Replacement of 2-arylethyl substituents at the piperidine ring nitrogen atom, resulted in sensitive changes of activity. Thus, N-methyl analog 99 is inactive at 100 mg/kg. N-benzyl analog 100 had greatly diminished activity (ED₅₀=10-45 mg/kg) as compared with its N-phenethyl analog Fentanyl (7) (ED₅₀=0.01 mg/kg) (Casy A F, Hassan M M, Simmonds B, Staniforth D. Structure-activity relations in analgesics based on 4-anilinopiperidine. J. Pharm. Pharmacol. 1969; 21(7):434-440, Srulevitch D B, Lien E J. 4-phenylamidopiperidines: synthesis, pharmacological testing and SAR analysis. Acta Pharm. Jugoslavica. 1991; 41(2):89-106). It is very important to mention, that this is not typical for the 4-phenylpiperidine series to which fentanyl is usually identified. Moreover, N-allyl substitution on the piperidine ring does not confer antagonist activity as in the case of morphinan derivatives. N-(1-propyl), N-(1-(2-phenoxyethyl)-, N-(1-(3-phenoxypropyl)- and N-(1-(2-cyanoethyl)-4-piperidinyl)-propionanilides (101), displayed activity similar to fentanyl (Casy A F, Hassan M M, Simmonds B, Staniforth D. Structure-activity relations in analgesics based on 4-anilinopiperidine. J. Pharm. Pharmacol. 1969; 21(7):434-440, Srulevitch D B, Lien E J. 4-phenylamidopiperidines: synthesis, pharmacological testing and SAR analysis. Acta Pharm. Jugoslavica. 1991; 41(2):89-106, Gupta P K, Yadav S K, Bhutia Y D, et al. Synthesis and comparative bioefficacy of N-(1-phenethyl-4-piperidinyl)propionanilide (fentanyl) and its 1-substituted analogs in Swiss albino mice. Med. Chem. Res. 2013; 22(8):3888-3896). Replacement of the nitrogen atom for oxygen, sulfur or carbon (102-104) resulted in loss of activity [Vardanyan R S, Unpublished Data]. Addition of a nitrogen atom into the second position of piperidine ring (105-106) also resulted in disappearance of activity (FIG. 14a) (Vartanyan R S, Gyul'budagyan A L, Khanamiryan A Kh, et al. Synthesis of 1-phenethyl-2-methyl- and 1-methyl-2-phenethyl-4-(N-propionylanilino)hexahydro pyridazines. Arm. Khim. Zh. 1987; 40(9):563-569). Replacement of amide carbonyl oxygen for sulfur via interaction of fentanyl with phosphorus pentasulfide preserves strong analgesic activity for N-(1-(2-phenylethyl) series of compounds (Jilek J, Rajsner M, Valenta V, et al. Synthesis of piperidine derivatives as potential analgesic agents. Coll. Czech. Chem. Comm. 1990; 55(7):1828-1853).

### Bivalent Ligands Based on Fentanyl

It is clear that activity at a single receptor is often insufficient and recent research, including in the pain area, is starting to be focused on ligands that have multiple activities. Multitargets (multivalent ligand) are believed to be ‘smarter’ than traditional drugs that primarily target a single receptor (Morphy R, Rankovic Z. Designed multiple ligands, an emerging drug discovery paradigm. J. Med. Chem. 2005; 48(21):6523-6543, Dietis N, Guerrini R, Salvadori C G, et al. Simultaneous targeting of multiple opioid receptors: a strategy to improve side-effect profile. Br. J. Anaesth. 2009; 103(1):38-49, Liu Z, Zhang J, Zhang A. Design of multivalent ligand targeting G-protein coupled receptors. Curr. Pharm. Des. 2009; 15(6):682-718, Schiller P W. Bi- or multifunctional opioid peptide drugs. Life Sci. 2010; 86:598-603, Shonberg J, Scammells P J, Capuano B. design strategies for bivalent ligands targeting GPCRs. ChemMedChem. 2011; 6:963-974, Balboni G, Salvadori S, Marczak E D, et al. Opioid biunctional ligands from morphine and the opioid pharmacophore Dmt-Tlc. Eur. J. Med. Chem. 2011; 46(2):799-803, Hruby V J, Porreca F, Yamamura H I, et al. New paradigms and tools in drug design for pain and addiction. AAPS J. 2006; 8(3):E450-E460). Attempts for creation of bivalent ligands based on fentanyl as a carrier of μ-opioid activity have been examined recently. The general idea of these studies is presented in FIG. 15a. The fundamental chemistry behind the work is focused on the creation of a plethora of new functionalized fentanyls able to undergo coupling reactions for the synthesis of bivalent ligands including peptide/non-peptide compounds. This approach gives the possibility to create a wide variety of μ-opioid/adjuvant analgesics bivalent ligands.

Newly synthesized types of functionalized μ-opioid derivatives for creation of bivalent ligands are presented in FIG. 16a. Among them are carboxy-(106) (R, Kumirov V K, Nichol G S, et al. Synthesis and biological evaluation of new opioid agonist and neurokinin-1 antagonist bivalent ligands. Bioorg. Med. Chem. 2011; 19(20):6135-6142) and amino-fentanyl (Petrov R R, Vardanyan R S, Lee Y S, et al. Synthesis and evaluation of 3-aminopropionyl substituted fentanyl analogs for opioid activity. Bioorg. Med. Chem. Lett. 2006; 16(18):4946-4950) derivatives (107), able to be involved in coupling reactions with amino acids and peptides, first of all intended for the synthesis of peptide/non-peptide compounds. Among them are ene-derivatives (108) (Van Dyke J W, Jr, Havera H J, Johnson R D, et al. Cardiovascular activity of some substituted 2-aminobenzoquinolizines. Med. Chem. 1972; 15(1):91-94), able to undergo a whole plethora of alkene chemistry and initially intended for thiol-ene ‘click’ reactions with cysteine derivatives. Allyloxy- and propargyl-oxy derivatives (109, 110) (Van Dyke J W, Jr, Havera H J, Johnson R D, et al. Cardiovascular activity of some substituted 2-aminobenzoquinolizines. J. Med. Chem. 1972; 15(1):91-94), are synthesized also for their use in the many possibilities of double and triple bond reactions, and first of all in ‘click’ reactions. Amino acid derivatives (112) (Maryanoff B E, McComsey D F, Taylor R J, et al. Synthesis and stereochemistry of 7-phenyl-2-propionanilidobenzo[a] quinolizidine derivatives. Structural probes of fentanyl analgesics. J. Med. Chem. 1981; 24(1):79-88) were prepared for further synthesis of 1-(piperidin-4-yl)piperazine congeners. Phenylhydrazines (112) (Kudzma L V, Evans S M, Turnbull S P, et al. Octahydro-1,2,3,4,4a,5,11,11a-pyrido3,4-c1,5benzoxazepines conformationally restricted fentanyl analogs. Bioorg. Med. Chem. Lett. 1995; 5(11):1177-1182) and hydrazines (115) (Nichol G S, Vardanyan R, Hruby V J. Synthesis and crystallographic study of N′-(1-benzylpiperidin-4-yl)acetohydrazide. J. Chem. Crystallography. 2010; 40(11):961-964) for the synthesis of propionylhydrazide analogs of fentanyl and variety of hetercyclizations to give indole, pyrazole and pyridazine derivatives. Finally, phenylalanine and isocyanato-derivatives (113, 114) (Van Dyke J W, Jr, Havera H J, Johnson R D, et al. Cardiovascular activity of some substituted 2-aminobenzoquinolizines. J. Med. Chem. 1972; 15(1):91-94) have been synthesized for ‘parallel transfer’ of adjuvant ligands from the propionyl-part of fentanyl to the 1-(2-phenethyl)-moiety.

The variety of newly created bivalent ligands based using the above mentioned functionalized fentanyls are presented in FIG. 17a. Synthesis of possible bivalent fentanyl/indomathacine (μ-agonist/COX inhibitor) compounds (84, 85, 87, 89) was described above (Kudzma L V, Evans S M, Turnbull S P, et al. Octahydro-1,2,3,4,4a,5,11,11a-pyrido3,4-c1,5benzoxazepines conformationally restricted fentanyl analogs. Bioorg. Med. Chem. Lett. 1995; 5(11):1177-1182).

We hypothesized that a bifunctional investigational tool could be compounds, pharmaceutical salts composed of μ-agonists and glial inhibitors (116), which could be prepared by an interaction of any opioid agonist as an organic base, in this case, fentanyl, with the classical glial inhibitor, fluorocitric acid, as an acid.

A possible advantage of this approach would be that after dissociation, both counterions will be able to act simultaneously and independently at their respective targets. However, since fluorocitric acid is not available commercially, we developed a large-scale method for the preparative synthesis of fluorocitric acid and its corresponding fentanyl salt (Vardanyan R, Kumirov V K, Hruby V J. Improved synthesis of d,l-fluoro-citric acid. J. Fluorine Chem. 2011; 132(11):920-924). The obtained compound was too toxic to continue studies in this direction.

Attempts to synthesize μ-agonist/NK1 antagonist bivalent compounds (118) combining fentanyl and well known NK1 antagonist (L-732138), by replacing the acetyl moiety in it for carboxy-fentanyl moiety have been realized, starting from carboxy-fentanyl derivatives (106) as the opioid part and tryptophan 3,5-bis(trifluoromethyl) benzyl ester as the NK1 antagonists part.

The large-scale synthesis of carboxy-fentanyls (106), started from reaction of 1-phenethyl-N-phenylpiperidin-4-amines (11) and appropriate acid anhydride. ‘Peptide chemistry’ methods with the carbodiimide activation of carboxyl function were successfully used for the synthesis of covalently bonded fentanyl/L-732138 pair (FIG. 18a) (R, Kumirov V K, Nichol G S, et al. Synthesis and biological evaluation of new opioid agonist and neurokinin-1 antagonist bivalent ligands. Bioorg. Med. Chem. 2011; 19(20):6135-6142). Another ‘universal method’ for creation of substituted carboxy-fentanyls was developed based on the idea of ‘isoimidium perchlorate chemistry’. The isoimidium perchlorates (117) were readily prepared from 106, successfully reacted with 1-tryptophan 3,5-bis(trifluoromethyl)benzyl ester and gave the desired 118. This method can be implemented for creation of any bivalent μ-/adjuvant analgesics with any other adjuvant containing any nucleophile group. The idea of ionic pair μ-agonist/NK1. antagonist compounds (119) has been realized in this case. The potassium salts (106a) were simply mixed with 1-tryptophan 3,5-bis(trifluoromethyl)benzyl ether hydrochloride followed by removal of the resulting KCl.

The biological studies indicate that the synthesized functionalized fentanyls (233) dramatically lose analgesic activity, which probably can be explained with the appearance of the ‘bulky’ carboxyl group with negative charge in the structure, and which is more or less restored by distancing of the carboxyl group, as well as by replacement of charged groups for covalently bonded derivatives. At the same time the behavior of tryptophan 3,5-bis(trifluoromethyl) benzyl ester does not change much. The data obtained indicate that both ionic and covalently linked bivalent ligands compete with high affinity for [³H] Substance P binding sites in recombinant CHO cells expressing the human NK1 receptor. Bivalent ligands with covalent linkage between the two pharmacophors exhibit reduced affinities, relative to the parent opioid (fentanyl) at the rat MOR and for [³H]DAMGO binding sites with very low affinities. In functional assays, all compounds, in which opioid and NK1 pharmacophores are bound covalently or by ionic bonds showed NK1 antagonism in vitro with no NK1 agonist activity up to 1 uM (R, Kumirov V K, Nichol G S, et al. Synthesis and biological evaluation of new opioid agonist and neurokinin-1 antagonist bivalent ligands. Bioorg. Med. Chem. 2011; 19(20):6135-6142).

Combinations of μ-opioid agonists with δ-opioid modulators are one of the most interesting and contradictory areas of analgesic studies. Some authors affirm that d-agonists increase antinociceptive responses to μ-agonists (Porreca F, Takemori A E, Sultana M, et al. Modulation of mu-mediated antinociception in the mouse involves opioid delta-2 receptors. J. Pharm. Exp. Therapeut. 1992; 263(1):147-152, Heyman J S, Vaught J L, Mosberg H I, et al. Modulation of mu-mediated antinociception by delta agonists in the mouse: selective potentiation of morphine and normorphine by [D-Pen2,D-Pen5]enkephalin. Eur. J. Pharm. 1989; 165(1):1-10, Heyman J S, Jang Q, Rothman R B, et al. Modulation of μ-mediated antinociception by delta agonists: characterization with antagonists. Eur. J. Pharm. 1989; 169(1):43-52). Others propose that “opioid compounds with mixed μ-agonist/δ-antagonist properties are expected to be analgesics with low propensity to produce tolerance and dependence” (Schiller P W, Fundytus M E, Merovitz L, et al. The opioid μ agonist/d antagonist DIPPNH 2[y] produces a potent analgesic effect, no physical dependence, and less tolerance than morphine in rats. J. Med. Chem. 1999; 42(18):3520-3526). A third group write that “ . . . μ-agonist signaling can be enhanced by cotreatment with δ-selective ligands including δ-selective antagonists” (Rozenfeld R, Devi L A. Receptor heterodimerization leads to a switch in signaling: β-arrestin2-mediated ERK activation by μ-d opioid receptor heterodimers. FASEB J. 2007; 21(10):2455-2465). Another group suggests that “μ-δ receptor interactions lead to profound modulation of μ receptor” (Gomes I, Gupta A, Filipovska J, et al. A role for heterodimerization of μ and δ opiate receptors in enhancing morphine analgesia. Proc. Natl Acad. Sci. USA. 2004; 101(14):5135-39). Finally, it is stated that “there is not only a differential localization of the δ- and μ- in dorsal root ganglion cells, but also that these differences underlie qualitatively distinct pain relieving profiles” (Scherrer C, Imamachi N, Cao Y-Q, et al. Dissociation of the opioid receptor mechanisms that control mechanical and heat pain. Cell. 2009; 137(6):1148-59). Interestingly, biphalin, a dimeric encephalin analog with both potent μ- and δ-agonist activity was shown to have highly potent analgesic activity and greatly reduced μ-opioid ligand toxicities and reduced tolerance (Horan P J, Mattia A, Bilsky E J, et al. Antinociceptive profile of biphalin, a dimeric enkephalin analog. J. Pharm. Exp. Ther. 1993; 265(3):1446-1454).

One of the early attempts for creation of μ-agonists/δ-agonists bivalent ligands with the fentanyl moiety was done via coupling aminofentanyl derivative 107 with known δ-receptor ligands from the enkephalin series [Tyr-D-Ala-Gly-Phe] to give compound 120 with good opioid affinity (1 nM at both δ- and μ-receptors) and bioactivity (34.9 nM in MVD and 42 nM in the GPI/LMMP bioassays) (FIG. 18a) (Petrov R R, Vardanyan R S, Lee Y S, et al. Synthesis and evaluation of 3-aminopropionyl substituted fentanyl analogs for opioid activity. Bioorg. Med. Chem. Lett. 2006; 16(18):4946-4950).

Another approach was carried out by implementing possibilities of carboxy-fentanyls (106) or the corresponding isoimidium salts (117) for linking different pharmacophores. In this case the dligand, a Tyr-D-Ala-Gly-Phe from the enkephalin series was bonded to a fentanyl ligand via a hydrazine bond, as had been done in biphalin. For that purpose 106 or 117 were condensed with protected phenylalanine hydrazide to give (121), followed with coupling with [Boc-Tyr(Boc)-d-Ala-Gly-OH] and final deprotection. Compounds (122) showed practically the same bioactivity as (120) in the range (48-69 nM in MVO and 42-44 nM in GPI/LMMP bioassays) (Van Dyke J W, Jr, Havera H J, Johnson R D, et al. Cardiovascular activity of some substituted 2-aminobenzoquinolizines. J. Med. Chem. 1972; 15(1):91-94).

A dramatic 100-fold increase in activity happened when the δ-ligand Dmt-substituted enkephalin-like structure Dmt-D-Yyy-Gly-Phe was ‘parallel transferred’ from the propionyl-part of fentanyl to the ‘modified’ 1-(2-phenethyl)-moiety. First attempts in this direction have been done by replacing the phenethyl group of fentanyl with several aromatic ring containing aminoacids (Lee Y S, Nyberg J, Moye S, et al. Understanding the structural requirements of 4-anilidopiperidine analogs for biological activities at μ and δ opioid receptors. Bioorg. Med. Chem. Lett. 2007; 17(8):2161-2165, Lee Y S, Petrov R, Kulkarni V, et al. Development of μ/d opioid ligands: enkephalin analogs containing 4-anilidopiperidine moiety. Adv. Exp. Med. Biol. 2009; 611:517-518). Obtained compounds showed broad (47 nM to 76 μM) and μ-selective activities. For the synthesis of possible μ/δ compounds (FIG. 19a) amine (5) was coupled with protected phenylalanine or its p-F(Cl)-substituted analogs to give (123), which after deprotection were stepwise transferred to desired bivalent ligands (125) (Lee Y S, Petrov R, Park C K, et al. Development of novel enkephalin analogs that have enhanced opioid activities at both μ and δ opioid receptors. J. Med. Chem. 2007; 50(22):5528-5532, Lee Y S, Kulkarani V, Cowell S M, et al. Development of potent μ and δ opioid agonists with high lipophilicity. J. Med. Chem. 2011; 54(1):382-386). Compounds with X═H and D-Yyy-Ala showed potent binding affinities (0.4 nM) at μ and δreceptors with increased hydrophobicity (a Log P=2.96), and potent agonist activities in the MVD (1.8 nM) and GPI (8.5 nM) assays (Lee Y S, Petrov R, Park C K, et al. Development of novel enkephalin analogs that have enhanced opioid activities at both μ and δ opioid receptors. J. Med. Chem. 2007; 50(22):5528-5532). Ligands with X═F, D-Yyy-Nle showed potent subnanomolar opioid agonist activity (IC₅₀ values of 0.37 and 0.26 nM in the MVD and GPI assays, respectively) with excellent efficacy (EC₅₀=0.07 nM, Eₘₐₓ=48% at hDOR; EC₅₀=0.29 nM, Eₘₐₓ=98% at rMOR) at both receptors. Binding at the μ receptor was subnanomolar (Kᵢ=0.02 nM), and the Eₘₐₓ at the rMOR in the [35S]GTP-γ-S binding assay was 98%. These results suggest that halogen substitution (X═F, Cl) increases cell permeability and enhances potency. The effects are more pronounced for the μ receptor, and produce very well balanced (IC₅₀/IC₅₀ μ) mixed agonist activities for both receptors, respectively (Lee Y S, Kulkarani V, Cowell S M, et al. Development of potent μ and δ opioid agonists with high lipophilicity. J. Med. Chem. 2011; 54(1):382-386).

Dsuvia, is a tablet form of sufentanil (FIG. 20a), a synthetic opioid that has been used intravenously and in epidurals since the 1980s. It is 10 times stronger than fentanyl, also produced illegally in forms that have caused tens of thousands of overdose deaths in recent years.

Additional examples of compounds on substances contemplated as treatable by the invention as an antidote include the following, sometimes referred to as designer opiates and/or opioids, including, but not limited to, 3-Methylbutyrfentanyl, 3-MBF, 3-Methylfentanyl, 3-MF, 4-Chloroisobutyrfentanyl, 4-CliBF, p-CliBF, 4-Fluorobutyrfentanyl, 4-FBF, p-FBF, 4-Fluoroisovutyrfentanyl, 4-FiBF, p-FiBF, 4-Methoxybutyrfentanyl, 4-MeO-BF, p-MeO-BF, 4-Fluorofentanyl, 4-FF, p-FF, Acetylfentanyl, AF, Acrylfentanyl, AD-1211, AH-7921, α-Methylfentanyl, “China White”, Butyrfentanyl, BF, Desmethylprodine, MPPP, Furanylfentanyl, Fu-F, 4′-Nitromethopholine, MT-45, Nortilidine, O-Desmethyltramadol, U-5175^{[62]}, U-47700, U-77891, Valerylfentanyl, VF, W-15^{[63]}, W-18.

In a preferred embodiment, apparatus for applications and compositions are provided for buccal or nasal administration for treatment of patients suffering from opiate and/or opioid over-dosage such as any of the one or more compositions/compounds mentioned above.

With respect to a preferred aspect treatment of the invention addicts of opiates and opioids such as heroin and methadone sometimes suffer respiratory failure as a result of administration of an excessive dose of the drug. While opiate antagonists may be given to reverse severe opiate respiratory depression, the standard method of administration is by intravenous injection, which is difficult for a medically unskilled person to carry out successfully, particularly in the stress of an emergency situation. For simplicity of discussion, reference herein to opiate antagonists shall mean both opiate and opioid antagonists.

In a preferred aspect, the present invention seeks to provide systems of administering an opiate antagonist which can be carried out by an unskilled person rapidly and with a good chance of successfully reviving a patient suffering from opiate over-dosage. The compositions of the invention have the advantage that they can be administered by a first-aider or person having no medical training, such as a friend or neighbor of an addict. A single close of the antagonist can readily be sprayed into the nose or mouth of an addict who is having difficulty breathing, while undertaking standard resuscitation procedures. If the patient does not respond to the initial dose, further doses of the antagonist can be given until reversal of the opioid and/or opiate depression is apparent. An advantage is that treatment can be given quickly and effectively without the need for the first-aider to find a blood vessel and give an intravenous injection. Another advantage of the applicators of the invention is that they cannot be misused to give injections of other drugs and are thus more likely to be retained and used for their intended purpose.

The present invention may be embodied in many other specific forms employing any one or more of the pharmaceutically active, or active pharmaceutical ingredients (“API”) or bioactive substances and/or agents mentioned herein above to combat and/or treat substance addictive and/or behavioral disorder persons, impulse and urges including a myriad of suitable and/or effective application apparatus therefor without departing from the spirit or essential attributes thereof, and may include any known, and as yet unknown, substances and/or agents for combating and treating substance addicted and/or behavioral disordered persons.

According to one aspect of the present invention there is provided a spray applicator having a solution of an opiate and/or opioid antagonist selected from naloxone and/or naltrexone provide in a reservoir. The applicator is designed to be capable of delivering single or multiple doses of an efficacious amount of the antagonist from the reservoir, and the applicator portion preferably comprises a projecting delivery portion shaped and dimensioned for introduction into the nose or mouth of a patient.

According to another aspect of the invention there is provided a pharmaceutical composition for oral or nasal administration comprising an opiate and/or opioid antagonist, the composition being comprised in finely-divided solid form and comprising a water-susceptible solid carrier and the antagonist compound.

In a further preferred embodiment, naltrexone is preferred for use against fentanyl and its various derivatives and analogs as naltrexone has an active metabolite participating in the pharmacological response in reversing fentanyl and fentanyl analog overdose. Naloxone does not, and is much shorter acting. The naltrexone metabolite has a much longer half-life than the parent drug and therefore pharmacological effect of naltrexone can be prolonged The pharmacokinetic profile of naltrexone suggests that naltrexone and its metabolites may undergo enterohepatic recycling. Thus, naloxone often times requires renarcotization or repeated dosing or simply will not work to reverse overdose result in unnecessary deaths when other efficacious reversal drugs are available, specification naltrexone.

Naloxone is less effective or not effective at all in saving people who have overdosed on fentanyl because fentanyl binds more tightly than heroin to opioid receptors in the brain, so it is more difficult for naloxone to displace it. Multiple doses are frequently needed or even do not successfully reverse fentanyl overdoses such as in the case of musician Prince receiving medication of Narcan (naloxone) in the days before his death but it did not work. It is misleading when published articles or journals, particularly available on internet, convey a message that naloxone is fail-safe to treat fentanyl overdose while it is evident that naloxone does not always work. Such finding prompts researchers to look for alternatives to naloxone. The inventor found that naltrexone has a stronger binding affinity to the receptor than fentanyl and its derivative/analogue and naltrexone blocks the effects of fentanyl and carfentanyl by competitive bind at opioid receptors. The pharmacological effect together with prolonging effect capacity of naltrexone greatly help displace fentanyl and/or its derivative/analogue such as carfentanyl effectively without administrating massive multiple doses.

In a still further preferred embodiment, the spray applicator may be designed for dispensing the solution into the mouth, e.g. sub-lingually, and be provided with a projecting delivery portion for this purpose. An additionally preferred embodiment, the applicator is provided with a delivery portion which is shaped and dimensioned for introduction into a nostril so that the dose is sprayed directly into the nasal passages, and which method of administration may be more convenient and enables resuscitation to be continuously and simultaneously applied. Such a device which has such a projecting delivery portion is also advantageous it may be applied directly into the mouth. Suitable spray applicators may be single trip devices, and normally incorporate a pump or syringe action for forcing an amount of the solution of the antagonist compound out of a nozzle, or calibrated to administer a premeasured amount of antagonist.

According to the aspect of the invention in which the pharmaceutical composition is in powder form, it is preferably administered nasally. In this embodiment, the composition is packaged via a dispenser having a projecting portion for introduction into a nostril, and preferably in calibrated measured amounts. Normally, a propellant is employed for generating an aerosol of the powdered pharmaceutical in a stream of gas. The dispenser will generally include calibration means for metering doses of the composition dispensed into the patient's nasal passages.

Preferred opiate and/or opioid antagonists for use in the compositions of this invention include naloxone (17-allyl-6-deoxy-7,8-dihydro-14-hydroxy-6-oxo-17-normorphine), and naltrexone, (17-(cyclopropylmethyl)-4,5α-epoxy-3,14-djτydroxymorphinan-6-one.), although any efficacious antagonist and/or agonist is contemplated for use in this invention, or any anti-addiction and efficacious behavioral disorder treatment substance in any effective treatment combination is contemplated for use herein.

Naloxone has a high affinity for u-opioid receptors in the central nervous system, and is a u-receptor competitive antagonist, and a pure antagonist with no agonist properties. Naloxone has long been used to counter the effects of opiate overdose, such as heroin or morphine, and specifically life threatening depression of the central nervous system, respiratory system, and is also used to treat hypotension. Nalaxone is also combined with buprenorphine in a drug composition called Suboxone which is used to treat opiate addiction.

Enteral naloxone has also been used with opiate therapy in mechanically ventilated acute care patients in the reduction of gastritis and esophagitis. Additionally, a combination of oxycodone and naloxone has been used for the prophylaxis of opiate induced constipation in patients requiring strong opiate therapy, and known as Targin and Targinact. A variant of naloxone known as (+)-nalaxone has also been used in treating opiate-related addiction, in binding to TLR4 immune receptors and inhibiting production of dopamine responsible for substance addiction, but still retaining pain relieving effect. For example, in the simultaneous administration of morphine and (+)-nalaxone, the intended analgesic effect of morphine will be present but without the potential for morphine addiction.

Naltrexone is an opiate receptor antagonist with qualitatively different effects than naloxone, and is most commonly known as useful in dependence treatment, rather than in emergency overdose treatment in reversibly blocking and attenuating the effects of opiates. Using naloxone in place of naltrexone is known to cause acute opiate withdrawal symptoms, and conversely using naltrexone in place of naloxone in a possible overdose situation is known to possibly lead to insufficient opiate antagonism and failure to reverse the overdose. Naltrexone is primarily known to help patients overcome opiate addiction by blocking euphoric effects. Naltrexone is also well known in its effective treatment of alcohol dependence and for its efficacy in reducing frequency and severity of relapse to drinking. Naltrexone also has been shown to reduce heavy drinking when used in people who continue drinking while taking naltrexone, and may even have increased efficacy in alcohol consumption treatment when used during active drinking rather than during abstinence, known as the Sinclair Method. Other known uses for naltrexone include treatment of depersonalization disorder, and in low doses (“low-dose naltrexone, or LDN”) used for treating non-chemical dependency maladies such as multiple sclerosis, fibromyalgia, and even forms of cancer and HIV, and in self-injurious behaviors, often times present in persons with developmental disabilities, such as autism, to inhibit the release of beta-endorphin which binds to the same receptors as opiates, such as heroin and morphine. Natrexone is also known to be useful in treatment of impulse control disorders, such as compulsive gambling, theft or kleptomania, pornography addiction and compulsive hair pulling, or trichotillomenia.

In some preferred embodiments herein a mixture of two or more anti-addiction or behavioral disorder treatment substances, such as two or more opiate antagonists may (or opiate or opiate/opioid antagonists, as the case may be) be employed. In the use of antagonists, preferably, naloxone may be used as a spray-able liquid composition and naltrexone may be used in the form of a powdered, solid composition, usually for nasal administration, such as in a spray particulate mist application along with sprayable liquid use as well. Naltrexone in a spray form in a dosage range as discussed herein is preferred as a deployable spray/atomized delivery herein for several reasons, although any substance abuse treatment compound or urge treatment compound in any physical mode is contemplated for use herein. In some embodiments, the combination of naltrexone and naloxone can be employed. In some further embodiments, the admixture of opioid/opiate antagonists may include naltrexone and buprenorphine. In still further embodiments, mixture of naltrexone, naloxone and buprenorphine can be used. The formulation for nasal and oral delivery of compositions according to die invention contain amount of naltrexone greater than 1.0% (w/v), and preferably about 3-5 mg/injection, and/or pharmaceutically efficacious amount of naloxone and/or pharmaceutically efficacious amount of buprenorphine.

Naltrexone in a spray form in a dosage range as disclosed herein is preferred for use due to its longer duration of efficacy and longer potency. This compound when administered will not wear off quickly, and will most likely not have to be repeatedly administered such as the less potent naltrexone charges, and, for example, naloxone currently in wide spread conventional use. A dose of naltrexone, compared to naloxone, is preferred due to the greater potency of naltrexone. Naltrexone is also preferably used herein to precipitate withdrawal such that a patient may be treated with additional naltrexone in various forms, such as in implanted pellet form or an injectable form, to maintain abstinence.

Notwithstanding, any drug compound that is absorbed subcutaneously and/or any medication that can be administered subcutaneously is contemplated for use herein as a spray administered compound including, adrenaline, and/or epinephrine, as a non-selective adrenaline agonist to potentially reverse the effects off allergens as well as substances of abuse and/or an adverse reaction to any compound.

It has been found, surprisingly, in accordance with one aspect of this limitation that it is possible to administer naltrexone in the form of liquid solution at low doses but yet higher than conventionally taught by the nasal and/or buccal route with excellent results both for attenuation of the undesirable side-effects due to the administration of opioids and in the case of excessive ingestion thereof.

Low dose according to the invention means a dose above 1% (w/v) and preferably 3-5 mg/injection.

Thus, the liquid formulations for nasal administration according to the invention contain amounts of naltrexone (normally in the form of hydrochloride salt) greater than about 1.0% (w/v), and preferably about 3-5 mg/injection.

As used herein, the terms “about” or “approximately” broaden the numerical value. For example, in some cases, “about” or “approximately” refers to +/−10%, of the relevant unit value. Also, the disclosure of ranges is intended as a continuous range including every value between the minimum and maximum values recited.

As used herein, the term “treatment” embraces all the different forms or modes of treatment as known to those of the pertinent art and in particular includes preventative, curative, delay of progression and palliative treatment,

Unless otherwise specified, values expressed as % refer to % w/v.

As used herein, the term “opioid receptor antagonist” includes any substance that selectively blocks an opioid receptor of any type (e.g., mu, delta, kappa, etc,) or subtyped (e.g., mu1/mu2). Suitable opioid receptor antagonists for use in the present invention include, but are not limited to, any centrally acting opioid receptor antagonist. In some embodiments, the antagonist is selected from naltrexone, nalmefene, naloxone, naloxonazine, nor-binaltorphimine, the opioid receptor antagonist is naltrexone.

The term “subject”, or “patient”, refers to an animal, for example, a mammal, such as a human, who is the object of treatment. The patient may also be a domestic production animal, exotic zoo animal, wild animal, or companion animal. The subject, or patient, may be either male or female.

Opioid receptor agonists (sometimes abbreviated as opioid agonists, or opioids) and opioid receptor antagonists (sometimes abbreviated as opioid antagonists) can also be called opiates.

The opioid receptor antagonist may be in free form or in pharmaceutically acceptable salt or complex form.

The composition is administered transmucosally to the patient. In some embodiments, the transmucosal administrations is selected from intranasal, buccal, sublingual, vaginal, ocular and rectal route (such as suppository) of administration. In one specific embodiment, the composition is administered sublingually to the patient. In a preferred embodiment, the composition is administered intranasally to the patient.

The pharmaceutical composition is also suitable for the parenteral administrations such as intravenous (injection into a vein), subcutaneous (injection under the skin), intramuscular (injection into a muscle), inhalation (aerosols, infusion through the lungs), and percutaneous (absorption through intact skin) and ophthalmic. For ophthalmic administration, polyhydric alcohols may be incorporated into aqueous solutions of naloxone hydrochloride to give ophthalmically acceptable formulations with enhanced storage stability relative to previously proposed ophthalmic formulations such as buffered or saline solutions, for example such that the naloxone content exhibits minimal degradation over periods of six months to one year or more.

The liquid solutions according to the invention are normally aqueous solutions or aqueous-alcoholic solutions in which the alcohol is preferably ethanol, and preferably in an amount to deliver a dosage amount greater than 1 mg, and preferably about 3-5 mg per administration. In addition, the solutions contain a buffer, the purpose of which is to maintain the pH at the value at which the opioid antagonist is in the form of a salt, for example as hydrochloride. The buffers may be selected from the following: citric acid/sodium citrate, citric acid/sodium hydroxide, dibasic sodium phosphate/citric acid, dibasic sodium phosphate/monobasic potassium phosphate, acetic acid/sodium acetate. The excipients used for the compositions of this type may also comprise antimicrobial preservatives, agents that increase the tonicity and agents that increase the viscosity of the solution (viscosity improvers). Oil based compositions and various suspensions, as known in the art are also contemplated for use herein.

Among the antimicrobial preservatives, such compounds may include, for example, benzalkonium chloride, methylparaben, propylparaben, sodium benzoate, benzoic acid, phenylethyl alcohol or mixtures thereof; preferably in amounts between 0.005-0.50% (w/v), preferably 0.005-0.30% (w/v), more preferably 0.01-0.1% (w/v).

Agents that increase tonicity are, for example, sodium chloride, dextrose, lactose or mixtures thereof; such as, for example, in amounts between 0.1-5.0% (w/v), preferably 0.1-2.0% 0 (w/v), more preferably 0.1-0.9% (w/v).

Viscosity improvers may be selected from, for example, hydroxypropyl methylcellulose (hypromellose), hydroxyethyl cellulose, hydroxypropyl cellulose, methylcellulose, microcrystalline cellulose, carboxymethylcellulose sodium, xanthan gum or mixtures thereof; such as, for example, in amounts between: 0.01-2.0% (w/v), preferably 0.02-1.0% (w/v), more preferably 0.05-0.5% (w/v).

The formulations according to the invention may be, by example, prepared following the standard techniques employed for preparing solutions for nasal application.

The following may be, for example, dissolved in a given amount of water: the preservative, the salts for the buffer, the agent for increasing osmolality, and then the viscosity improver. When the solution obtained is clear, the active ingredient is dissolved therein and the solution made up to the required volume with water.

Where the antagonist is in the form of a liquid composition, it may be a solution in a pharmaceutically acceptable carrier or co-solvent such as water or an alcohol, such as ethanol e.g. an aqueous solution containing an appropriate percentage of ethanol. Naloxone and naltrexone are both freely soluble in water and aqueous alcohol when in the form of a salt, such as a hydrochloride. Alternatively, the opiate antagonist may be dissolved in dilute saline solution, e.g. approximately isotonic salt solution. The composition may include a buffering agent to maintain the opiate in solution in the salt form, e.g. a phosphate buffer, such as sodium hydrogen phosphate to maintain the solution at a slightly acidic pH. A solution of the antagonist, usually in the form of the hydrochloride, for example, at a concentration greater than 1% by weight, preferably 3-5 mg-per administration in spray form be employed for nasal or buccal administration in accordance herein. The liquid composition may be packaged in a calibrated metered dosage spray dispenser, using a pump or propellant.

In the case of a solid, powdered composition for nasal administration, the antagonist is mixed with one or more solid, powdered carriers. Suitable carriers include saccharides such as sorbitol, mannitol, lactose, fructose, glucose and sucrose. Other carriers include water-soluble or swellable polymers such as cellulose derivatives, for example, hydroxypropyl methyl cellulose and carboxymethyl cellulose. A solid salt of the antagonist, e.g. the hydrochloride, maybe mixed with a carrier, or coated with the carrier or with a third material such as a hydrophilic polymer.

The solid, powdered composition containing the opiate antagonist may be packaged in a dispenser with a suitable propellant, such as HFC-134a or HFC-227. Again, a valve may be provided, which is adapted to dispense a dosage unit of the antagonist, or medication of about greater than 1 mg per administration preferably 3-5 mg per administration in accordance with this invention.

It may also be desirable to include an anti-oxidant, such as ascorbic acid or citric acid in the powdered formulation.

The compositions of the invention may also include antiparkinson compounds or drugs to treat or relieve the effects of Parkinson's disease like symptoms including, but not limited to, L-Dopa, Deprenyl, Tyrosine Hydroxylase, Apomorphine, and Anticholinergic drugs.

The compositions of the invention may also include any one or more of common additives, adjuvants, excipients, coloring agents, thickeners and the like commonly found in any pharmaceutical formulation or composition and/or found in neutraceutical compositions.

The invention is illustrated by the following Examples of pharmaceutical compositions suitable for use in dispensing an opiate antagonist in accordance with the invention and by the accompanying drawing, and description of one form of spray applicator in accordance with a preferred embodiment of the invention for dispensing a liquid composition.

### EXAMPLE 1

Sprayable Aqueous Liquid Composition for a Nasal Applicator.

Naloxone hydrochloride was dissolved in a solution of purified water to form a solution containing 0.8% weight/volume of the naloxone. Benzalkonium chloride was added to the hydrochloride solution in an amount of 0.025% weight/volume as a preservative. The solution may be buffered to a pH of about 6.5 using a phosphate buffer (sodium or potassium hydrogen phosphate). The solution was packaged into a dispenser as shown in the accompanying drawing, giving a shot volume of 50 μl (micro litre) which is equivalent to a unit dose of 400 μg (microgram) per shot.

### EXAMPLE 2

Solid, Powdered Nasal Preparation.

Powdered solid naloxone hydrochloride was mixed with powdered dextrose or lactose in an amount of from 2% weight/volume naloxone HCl and 98% weight/volume of the finely powdered sugar. The resulting mixture can be subsequently coated with a vinyl pyrollidone to form a free-flowing powder in which the opioid antagonist is present in a concentration of 2% by weight. The powdered composition is packaged in a dispenser, for example, as described in WO 99/27920.

### EXAMPLE 3

Naloxone HCl was dissolved in water with mannitol or lactose in a weight ratio of 2:98. The resulting solution was spray dried or freeze dried to form a fine powder containing 2% of naloxone HCl.

The powdered product can be packaged in an aerosol can with a low boiling propellant fitted with a metering valve or in a dispenser as described, for example, in WO 99/27920.

In some embodiments, a kit may be provided with an atomizer attached to a reservoir containing a liquid with the active ingredient/ingredients naltrexone, mixture of naltrexone and naloxone, or mixture of naltrexone, naloxone and buprenorphine with instructions for use. In some embodiments, a kit may be provided and comprise one or more single dose containers filled with the active pharmaceutical ingredient (“API”) composition, a sheet of instructions, and one or more mucosal atomization devices (MADs). In some embodiments, one MAD is pre-fitted to each single dose container. In some embodiments, the kit comprises one or more MADs capable of being fitted to the single dose container(s) prior to use. In some embodiments, the MAD can be fitted to a syringe, or an MAD with syringe can be used in conjunction with a filled vial. Any MAD capable of being fitted to a syringe, e.g., fitted with a luer lock, can be employed. MADs are available commercially and include LMA/MAD Nasal™ intranasal mucosal atomization device (LMA North America, Inc., Sand Diego, Calif.) and Wolfe-Tory Mucosal Atomization Device MAD (Wolfe-Tory Medical, Salt Lake City, Utah).

In an additional example device, an applicator may comprise a body part moulded from a flexible plastic material and having a projecting part suitably sized for insertion into a nostril. The projecting part has an internal tube, which extends from a tip to approximately a junction between the projecting part and a main body part. At its distal end, a tube may be joined to the inside of the projecting part, e.g. by forming part of an integral moulding, and communicate with a discharge orifice. A solution of the drug or API compound to be dispensed is contained in reservoir which is preferably made from transparent plastic or glass so that it can be seen by inspection if it contains any API or approximately how much API it does contain. For this purpose, the solution may be colored with a pharmaceutically acceptable dye.

A piston is made from flexible plastic material (e.g. polythene) and carries a solid piston rod which is formed with a passage. The passage communicates with the interior of the reservoir and terminates in a cross bore. The assembly consisting of the reservoir and piston and piston rod may be fitted into the body of the applicator by introducing the rod into the tube. The rod is a free fit into the part of the tube nearest to main body part but is a tighter fit into the distal end of the tube. With the projection parts situated in the patient's nostril, pressure is applied to the free end of the reservoir, e.g. by placing the fingers on the surfaces of the device and the thumb on the end of the reservoir and squeezing. This forces liquid with API from the reservoir along a passage, out of the cross bore and into the tube. Continued pressure forces liquid in a spray out of the orifice by the rod acting as a piston in the tube. The tube may be tapered slightly towards the orifice so that higher pressure can be developed within its distal end. It will be appreciated that by shaping the projecting part as a tapering fit in the nostril, a major amount of the API composition is retained in the nasal passages or within the mouth.

Although the invention herein has been described with reference to particular embodiments, it is to be understood that these embodiments are for illustrative purposes only. The present invention may be embodied in many other forms employing any of the pharmaceutical active agents or API's mentioned herein without departing from the spirit and scope of the invention as defined in the appended claims.

1. A method to treat carfentanyl overdose comprising administrating a pharmaceutical formulation in the form of liquid solution for spray administration by the nasal and/or buccal route containing naltrexone as active ingredient in an amount effective to reverse the effects of carfentanyl overdose.

2. Method according to claim 1 characterized in that the naltrexone is in an amount greater than about 1%.

3. Method according to claim 1, wherein said liquid solutions are aqueous or aqueous-alcoholic solutions.

4. A method to treat carfentanyl overdose comprising administrating a pharmaceutical formulation in the form of liquid solution for spray administration by the nasal and/or buccal route containing naltrexone in an amount greater than 1% and a pharmaceutically efficacious amount of naloxone as active ingredients.

5. Method according to claim 4 characterized in that the naltrexone is in an amount of about between 3-5 mg/injection.

6. Method according to claim 4, wherein said liquid solutions are aqueous or aqueous-alcoholic solutions.

7. A method to treat carfentanyl overdose comprising administrating a pharmaceutical formulation in the form of liquid solution for spray administration by the nasal and/or buccal route containing naltrexone in an amount greater than 1%, pharmaceutically efficacious amounts of naloxone and buprenorphine as active ingredients.

8. Method according to claim 7 characterized in that the naltrexone is in an amount of about between 3-5 mg/injection.

9. Method according to claim 7, wherein said liquid solutions are aqueous or aqueous-alcoholic solutions.

10. A carfentanyl overdose antidote kit comprising one or more single dose containers filled with a pharmaceutical formulation in the form of liquid solution for spray administration by the nasal and/or buccal route containing naltrexone as the active ingredient in an amount effective to reverse the effects of carfentanyl overdose, and optionally comprising a sheet of instructions and/or one or more mucosal atomization devices.

11. The kit according to claim 10 characterized in that the naltrexone is in an amount greater than about 1%.

12. The kit according to claim 10, wherein said liquid solutions are aqueous or aqueous-alcoholic solutions.

13. A carfentanyl overdose antidote kit comprising one or more single dose containers filled with a pharmaceutical formulation in the form of liquid solution for spray administration by the nasal and/or buccal route containing naltrexone in an amount effective to reverse the effects of carfentanyl overdose and a pharmaceutically efficacious amount of naloxone as active ingredients, and optionally comprising a sheet of instructions and/or one or more mucosal atomization devices.

14. The kit according to claim 13 characterized in that the naltrexone is in an amount greater than about 1%.

15. The kit according to claim 13, wherein said liquid solutions are aqueous or aqueous-alcoholic solutions.

16. A carfentanyl overdose antidote kit comprising one or more single dose containers filled with a pharmaceutical formulation in the form of liquid solution for spray administration by the nasal and/or buccal route containing naltrexone in an amount effective to reverse the effects of carfentanyl overdose, pharmaceutically efficacious amounts of naloxone and buprenorphine as active ingredients, and optionally comprising a sheet of instructions and/or one or more mucosal atomization devices.

17. The kit according to claim 16 characterized in that the naltrexone is in an amount greater than about 1%.

18. The kit according to claim 16, wherein said liquid solutions are aqueous or aqueous-alcoholic solutions.

19. The kit according to claim 10 wherein the reversal of the effects of carfentenyl overdose last without renarcotization upon application of one dose of 1-3 mg of naltrexone.
